(19) United States
(12) Patent Application Publication
(10) Pub. No.: US 2022/0229071 A1
(43) Pub. Date: Jul. 21, 2022

(54) DIAGNOSTIC AND PROGNOSTIC METHODS FOR PERIPHERAL ARTERIAL DISEASES, AORTIC STENOSIS, AND OUTCOMES

(71) Applicant: PREVENCIO, INC., Kirkland, WA (US)

(72) Inventors: Rhonda Fay RHYNE, Kirkland, WA (US); Craig Agamemnon MAGARET, Seattle, WA (US); Grady BARNES, Kirkland, WA (US); James Louis JANUZZI, Wellesley, MA (US); John STROBECK, Allendale, NJ (US)

(21) Appl. No.: 16/760,889

(22) PCT Filed: Nov. 2, 2018
(86) PCT No.: PCT/US18/59080

§ 371 Date: Apr. 30, 2020

Related U.S. Application Data
(60) Provisional application No. 62/580,916, filed on Nov. 2, 2017.

(51) Int. Cl. G01N 33/68

(52) U.S. Cl. CPC G01N 33/6893; G01N 2800/32; G01N 2800/60; G01N 2800/50; G01N 2800/52; G01N 2800/323

(57) ABSTRACT

Compositions and methods are provided for diagnosis and/or prognosis of peripheral artery disease, aortic stenosis, or cardiovascular events in a subject. In some embodiments, the method includes measuring and comparing the level of particular proteins to other proteins. In other embodiments, the method includes comparison with clinical variable information.

### FIELD

The present disclosure relates protein marker panels, assays, and kits and methods for determining the diagnosis, monitoring and/or prognosis of a cardiovascular disease or outcome in a patient.

### BACKGROUND

There are a number of cardiovascular diseases afflicting humans. The prevalence of peripheral artery disease (PAD) is estimated to be 10%-25% in people aged ≥55 years and increases to approximately 40% in community populations aged >80 years [1, 2]. Approximately 4-8 million people are affected by PAD in the United States of America [3-5]. In Germany, around 1.8 million people have symptomatic PAD and each year between 50,000 to 80,000 patients develop chronic limb ischemia (CLI) [6, 7]. In a population-based study in Western Australia, the prevalence of PAD was reported to be approximately 23% in men aged 75-79 years [1]. Recent reports suggest that the burden of PAD has increased globally over the last decade [8-10]. Atherosclerosis-induced CLI has been associated with a mortality rate of 20%-25% in the first year after presentation and a survival rate of less than 30% at five years [11-14]. Previous reports suggest that CLI patients have a three-year limb amputation rate of about 40% [15-17]. Recurrent CLI due to the failure of lower extremity revascularization is associated with a poor outcome [18-19]. In the Bypass versus Angioplasty in Severe Ischemia of the Leg (BASIL, n=216) trial, the re-intervention rate in 216 patients with CLI treated by percutaneous transluminal angioplasty was 26% at 12 months [11]. Reasons for revascularization failure include restenosis, and residual and progressive atherosclerosis. Approximately 20%-30% of CLI patients are not ideal candidates for interventional procedures for a number of reasons such as the distribution of the occlusive disease and the patient's co-morbidities [20]. Patients with CLI represent a small subset of the total PAD population; however, the high incidence of CVD events, repeated requirement for medical attention and high amputation rates lead to significant health service costs associated with these individuals [21-22].

Aortic valve stenosis (AS) represents the most common type of acquired valve heart disease. Its incidence increases with age; from 3% to 9% of adults over 75 years of age develop aortic valve stenosis. The pathophysiological mechanisms of AS have been extensively studied. Progression of AS is characterized by a number of abnormalities in calcification regulation, inflammation/adipokine dysregulation, prothrombic state, and altered von Willebrand factor function. The current understanding of the mechanisms of AS involves a complex role of multiple cell types, in particular myofibroblasts and macrophages [23].

A need therefore exists for simple, reliable, yet novel, methods to improve the diagnosis and/or prognosis and/or monitoring of peripheral artery disease, aortic stenosis, and associated outcomes, including limb amputation.

### SUMMARY

In an aspect, provided herein are methods of determining or monitoring peripheral artery disease in a subject. The methods can include, for example, providing a biological sample from a subject suspected of having peripheral artery disease, applying the biological sample to an analytical device that is programmed to detect the concentration of at least two protein markers in the sample, normalize the concentrations against a quantification standard, and transform the normalized concentrations. The methods further can include optionally determining the status of at least one clinical variable, optionally entering the variable as a mathematical factor into the analytical device, calculating a diagnostic score using an algorithm based on the normalized, transformed concentrations of protein markers and optionally, the status of the clinical variable(s), classifying the diagnostic score as a positive, intermediate, or negative result, and determining peripheral artery disease in the subject as indicated by the diagnostic score. The at least two protein markers are selected from Table 1. The optional clinical variable(s) are selected from Table 2.

In an aspect, provided herein are methods of administering a therapeutic intervention to a subject suspected of having peripheral artery disease. The methods include (i) determining the subject's protein marker profile for a panel of at least two protein markers selected from Table 1; (ii) optionally, determining the status of at least one clinical variable for the subject, where the clinical variable is selected from Table 2; (iii) assigning a score to the subject based on the protein marker profile in (i) and optionally the clinical value status in (ii); and (iv) administering to the subject a therapeutic intervention based on the positive, intermediate or negative score. Provided in the methods herein, the score is selected from positive, intermediate, and negative, and the score is algorithmically derived from the normalized and mathematically transformed concentrations of protein markers in the subject's sample and optionally, the status of at least one clinical variable.

In an aspect, provided herein are methods of detecting two or more protein markers in a subject having hypertension and/or is suspected of having peripheral artery disease. The methods include selecting a subject that has hypertension and/or is suspected of having peripheral artery disease, providing a biological sample from the subject, applying the biological sample to an analytical device, and detecting the concentration of at least two protein markers selected from Table 1.

In an aspect, provided herein are panels for the diagnosis, monitoring and/or prognosis of peripheral artery disease. The panel includes target-binding agents that bind at least two protein markers selected from Table 1. The panel optionally includes at least one clinical variable selected from Table 2.

In an aspect, provided herein panels for the diagnosis of 50% or greater obstruction in a peripheral artery. The panel includes target-binding agents that bind protein markers for angiopoietin 1, eotaxin 1, follicle stimulating hormone, interleukin 23, kidney injury molecule 1, and midkine and includes the clinical variable of history of hypertension.

In an aspect, provided herein are methods of determining risk of peripheral limb amputation in a subject within a time period. The methods include providing a biological sample from a subject suspected of having a risk of peripheral limb amputation, applying the biological sample to an analytical device that is programmed to detect the concentration of at least two protein markers in the sample, normalize the concentrations against a quantification standard, and transform the normalized concentrations. The methods include optionally determining the status of at least one clinical variable, calculating a prognostic score using an algorithm based on normalized, transformed concentrations of protein markers and optionally, the status of the clinical variable(s), classifying the prognostic score as a positive, intermediate, or negative result, and determining the risk of peripheral limb amputation in the subject as indicated by the prognostic score. The at least two protein markers are selected from Table 1. The optional clinical variable(s) are selected from Table 2.

In an aspect, provided herein are methods of administering a therapeutic intervention to a subject suspected of having a risk of peripheral limb amputation. The methods include (i) determining the subject's protein marker profile for a panel of at least two protein markers selected from Table 1; (ii) optionally, determining the status of at least one clinical variable for the subject, where the clinical variable is selected from Table 2; (iii) assigning a score to the subject based on the protein marker profile in (i) and optionally the clinical value status in (i); and (ii) administering to the subject a therapeutic intervention based on the positive, intermediate or negative score. Provided in the methods herein, the score is selected from positive, intermediate, and negative, and the score is algorithmically-derived based on the normalized and mathematically transformed concentrations of protein markers in the subject's sample and optionally, the status of at least one clinical variable.

In an aspect, provided herein are methods of detecting two or more protein markers in a subject having diabetes mellitus type 2 and/or that is suspected of having a risk of peripheral limb amputation. The methods include selecting a subject that has diabetes mellitus type 2 and/or that is suspected of having a risk of peripheral limb amputation, providing a biological sample from the subject, applying the biological sample to an analytical device, and detecting the concentration of at least two protein markers selected from Table 1.

In an aspect, provided herein are panels for the prognosis of peripheral limb amputation. The panel includes target-binding agents that bind at least two protein markers selected from Table 1. The panel optionally includes at least one clinical variable selected from Table 2.

In an aspect, provided herein are panels for the prognosis of peripheral limb amputation within five years. The panels include target-binding agents that bind protein markers for kidney injury molecule-1 and vitamin D binding protein and includes determining the status of the clinical variable of history of diabetes mellitus type 2.

In an aspect, provided herein are methods of determining aortic valve stenosis in a subject. The methods include providing a biological sample from a subject suspected of having aortic valve stenosis, applying the biological sample to an analytical device that is programmed to detect the concentration of at least two protein markers in the sample, normalize the concentrations against a quantification standard, and transform the normalized concentrations into a score. The methods include optionally determining the status of at least one clinical variable, calculating a diagnostic score using an algorithm based on the normalized, transformed concentrations of protein markers and optionally, the status of the clinical variable(s), classifying the diagnostic score as a positive, intermediate, or negative result, and determining aortic stenosis in the subject as indicated by the diagnostic score. The at least two protein markers are selected from Table 1. The optional clinical variable is selected from Table 2.

In an aspect, provided herein are methods of administering a therapeutic intervention to a subject suspected of having aortic valve stenosis. The methods include (i) determining the subject's protein marker profile for a panel of at least two protein markers selected from Table 1; (ii) optionally, determining the status of at least one clinical variable for the subject, where the clinical variable is selected from Table 2; (iii) assigning a score to the subject based on the protein marker profile in (i) and optionally the clinical value status in (ii); and (iv) administering to the subject a therapeutic intervention based on the positive, intermediate or negative score. In embodiments, the score is selected from positive, intermediate, and negative, and the score is algorithmically-derived based on the normalized and mathematically transformed concentrations of protein markers in the subject's sample and optionally, the status of at least one clinical variable.

In an aspect, provided herein are panels for the diagnosis and/or monitoring of aortic stenosis, comprising at least two protein markers selected from those listed in Table 1 and optionally, at least one clinical variable selected from those set forth in Table 2.

In an aspect, provided herein are panels for the diagnosis of aortic valve stenosis, comprising fetuin A, N terminal prohormone of brain natriuretic peptide, and von Willebrand factor and the clinical variable of age.

### BRIEF DESCRIPTION OF THE DRAWINGS

FIG. 1 shows a receiver operating characteristic curve for the Prevencio PAD panel PAD158 (as described in Example 1) (N=353) to diagnose the presence of PAD (>50% obstruction in any peripheral vessel), and/or monitoring PAD progression or therapeutic effect. The panel had a robust cross-validated area under the curve (AUC) of 0.84 (not shown), and an in-sample AUC of 0.85 (shown in FIG. 1).

FIG. 2 shows a receiver operating characteristic curve for the Prevencio PAD panel PAD076 (as described in Example 2) (N=258) to diagnose the presence of PAD (>50% obstruction in any peripheral vessel), and/or monitoring PAD progression or therapeutic effect. The panel had a robust cross-validated area under the curve (AUC) of 0.72 (not shown) and an in-sample AUC of 0.76 (shown in FIG. 2).

FIG. 3 shows a receiver operating characteristic curve for the Prevencio panel AMPU018 (as described in Example 3) (N=353) for prognosis for Peripheral Limb Amputation Risk. The panel had a robust cross-validated area under the curve (AUC) of 0.84 (not shown) and an in-sample AUC of 0.87 (shown in FIG. 3).

FIG. 4 shows receiver operating characteristic curve for the Prevencio panel ASR025 (as described in Example 4) (N=1244) to diagnosis and monitor severe Aortic Valve Stenosis. The panel had a robust cross-validated area under the curve (AUC) of 0.74 (not shown) and an in-sample AUC of 0.76 (shown in FIG. 4).

FIG. 5 shows a 5-point score for diagnosis of peripheral artery disease and/or monitoring PAD progressions or therapeutic effect for the Prevencio PAD panel PAD158 (as described in Example #1).

FIG. 6 shows a 10-point score for diagnosis of peripheral artery disease and/or monitoring PAD progressions or therapeutic effect for the Prevencio PAD panel PAD076 (as described in Example #2). When the score was divided into low risk (score of ≤3/10) and high risk (score of ≥7/10) groups, we found NPV of 67% and PPV of 100% for obstructive PAD for each subgroup respectively.

FIG. 7 shows a Kaplan Meier curve for predicting revascularization within 1 year follow up (age and sex adjusted) for the Prevencio PAD panel PAD158; such risk extended to at least to 5 years (p<0.001) (as described in Example #1).

FIG. 8 shows a Kaplan Meier curve for predicting revascularization within 1 year follow up (age and sex adjusted) for the Prevencio PAD panel PAD076; such risk extended to at least to 5 years (p=0.002) (as described in Example #2).

### DETAILED DESCRIPTION

The practice of the technology described herein will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. [24-33].

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art.

As used herein, the singular terms “a”, “an”, and “the” include the plural reference unless the context clearly indicates otherwise.

Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment described herein. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2, or 1% to a reference quantity, level, value, concentration, measurement, number, frequency, percentage, dimension, size, amount, weight or length. In particular embodiments, the terms “about” or “approximately” when preceding a numerical value indicates the value plus or minus a range of 15%, 10%, 5%, or 1%.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The terms “disease” or “condition” refer to a state of being or health status of a patient or subject capable of being treated with the compounds or methods provided herein. The disease may be a cardiovascular disease. The disease may be an inflammatory disease. In some instances, the disease is peripheral artery disease. In some instances, the condition is peripheral limb amputation. In some instances, the disease is aortic stenosis. In some instances, the disease is diabetes mellitus type 2. In some instances, the disease is hypertension.

As used herein, the term “diagnosis” refers to an identification or likelihood of the presence of a cardiovascular disease or outcome in a subject.

As used herein, the term “prognosis” refers to the likelihood or risk of a subject developing a particular outcome or particular event.

As used herein, a “biological sample” encompasses essentially any sample type that can be used in a diagnostic or prognostic method described herein. The biological sample may be any bodily fluid, tissue or any other sample from which clinically relevant protein marker levels may be determined. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polypeptides or proteins. The term “biological sample” encompasses a clinical sample, but also, in some instances, includes cells in culture, cell supernatants, cell lysates, blood, serum, plasma, urine, cerebral spinal fluid, biological fluid, and tissue samples. The sample may be pretreated as necessary by dilution in an appropriate buffer solution or concentrated, if desired. Any of a number of standard aqueous buffer solutions, employing one of a variety of buffers, such as phosphate, Tris, or the like, preferably at physiological pH can be used.

“Treating” or “treatment” as used herein (and as well understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the risk or condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

The term “prevent” refers to a decrease in the occurrence of disease symptoms in a patient. The prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition. Non-limiting examples include humans, other mammals, bovines, rats, mice, dogs, monkeys, goat, sheep, cows, deer, and other non-mammalian animals. In some embodiments, a patient is human.

“Control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples). In some instances, the control is a quantification standard used as a reference for assay measurements. The quantification standard may be a synthetic protein, a recombinantly expressed purified protein, a purified protein isolated from its natural environment, a protein fragment, a synthesized polypeptide, or the like.

The term “cardiovascular disease” refers to a class of diseases that involve the heart or blood vessels. Cardiovascular disease includes, but is not limited to, coronary artery diseases (CAD), myocardial infarction (commonly known as a heart attack), stroke, hypertensive heart disease, rheumatic heart disease, cardiomyopathy, congestive heart failure, cardiac arrhythmias (i.e., atrial fibrillation, ventricular tachycardia, etc.), cerebrovascular disease, peripheral arterial disease, aortic valve stenosis, other cardiac valvular disease, and arterial thrombosis.

The term “cardiovascular event” as used herein denotes a variety of adverse outcomes related to the cardiovascular system. These events include, but are not limited to peripheral limb amputation, peripheral revascularization, coronary revascularization, myocardial infarct, heart failure, stroke, and cardiovascular death.

The term “peripheral artery disease” or “PAD” refers to a particular type of cardiovascular disease. PAD is characterized by narrowing or blockage of the arteries outside the heart and brain and includes, but is not limited to, supplying blood to the lower limbs, arms, and kidneys. Such obstruction may be clinically relevant at levels of 50% or greater, 60% or greater, 70% or greater, 80% or greater, 90% or greater, or 100%. PAD is principally caused by athero-thrombosis. PAD is a leading cause of morbidity due to the associated functional decline and limb loss. Both asymptomatic and symptomatic PAD are significant predictors of cardiovascular disease (CVD) events and mortality [34]. Current evidence suggests that PAD represents a CVD risk equivalent to or worse than coronary artery disease requiring aggressive medical management. The main recognized clinical presentations of PAD are intermittent claudication (IC) and critical limb ischemia (CLI). IC describes the symptoms of pain in the muscles of the lower limb brought on by physical activity which is rapidly relieved by rest. Critical limb ischemia (CLI) is a more severe manifestation of PAD, which presents as rest pain, ischemic ulceration or gangrene of the foot.

The term “peripheral revascularization”, also referred to as “percutaneous peripheral intervention”, “peripheral revascularization intervention”, “percutaneous peripheral revascularization,” or “peripheral bypass graft,” as used herein refers to the restoration of perfusion to a peripheral artery, including but not limited to the legs, that has suffered ischemia. It is typically accomplished through peripheral angioplasty (with a balloon and/or placement of a stent) or a peripheral bypass graft.

The term “peripheral limb amputation risk” as used herein refers to the prognosis of risk of limb amputation because of severe PAD. Patients with critical limb ischemia (CLI) have a high risk of limb loss and fatal or non-fatal vascular events, such as myocardial infarction (MI) and stroke [35]. Acute limb ischemia (ALI) occurs when there is a sudden interruption of blood flow to a limb typically due to an embolism or thrombosis [36]. In contrast to CLI, which typically develops over a prolonged period often preceded by IC, patients with ALI may not have preceding symptoms. ALI usually threatens limb viability more urgently than CLI possibly due to the absence of an established collateral blood supply to the limb.

“Aortic stenosis” or “AS” or “AoS”, also referred to as “aortic valve stenosis”, as used herein refers to a narrowing of the exit of the left ventricle of the heart (where the aorta begins), such that problems result. It may occur at the aortic valve as well as above and below this level. It typically gets worse over time. Symptoms often come on gradually with a decreased ability to exercise often occurring first. If heart failure, loss of consciousness, or heart-related chest pain occurs due to AS, the outcomes are worse. Loss of consciousness typically occurs with standing or exercise. Signs of heart failure include shortness of breath especially when lying down, at night, or with exercise, and swelling of the legs.

In the early stage of AS, initial AS plaque resembles the plaque of coronary artery disease (CAD). Subsequent studies also found that CAD and AS shared similar risk factors (37). Risk factors and mediators leading to calcific AS, such as older age, male sex, hypercholesterolemia, arterial hypertension, smoking, and diabetes, are also similar to those recognized as classic risk factors for vascular atherosclerosis.

However, there are significant differences between vascular atherosclerosis (more unstable process) and aortic valve (valve) calcification (more stable process). In the progression of CAD, plaque rupture is the major event leading to clinically relevant events, whereas in AS, it is progressive calcification, even with lamellar bone formation that causes immobility of the valve (38). In addition, the significance of several biological and clinical differences between CAD and AS cannot be excluded.

As described herein, the terms “marker”, “protein marker”, “polypeptide marker,” and “biomarker” are used interchangeably throughout the disclosure. As used herein, a protein marker refers generally to a protein or polypeptide, the level or concentration of which is associated with a particular biological state, particularly a state associated with a cardiovascular disease, event or outcome. Panels, assays, kits and methods described herein may comprise antibodies, binding fragments thereof or other types of target-binding agents, which are specific for the protein marker described herein.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. In various embodiments, detecting the levels of naturally occurring protein marker proteins in a biological sample is contemplated for use within diagnostic, prognostic, or monitoring methods disclosed herein. The term also includes fusion proteins, including, but not limited to, naturally occurring fusion proteins with a heterologous amino acid sequence, fusions with heterologous and homologous leader sequences, with or without N-terminal methionine residues; immunologically tagged proteins; and the like. The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The term “antibody” herein is used in the broadest sense and specifically covers, but is not limited to, monoclonal antibodies, polyclonal antibodies, multi-specific antibodies (e.g., bispecific antibodies) formed from at least two intact antibodies, single chain antibodies (e.g., scFv), and antibody fragments or other derivatives, so long as they exhibit the desired biological specificity. The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The term “monoclonal antibody” as used herein refers to an antibody obtained from a population of substantially homogeneous antibodies, i.e., the individual antibodies comprising the population are identical except for possible naturally occurring mutations that can be present in minor amounts. In certain specific embodiments, the monoclonal antibody is an antibody specific for a protein marker described herein.

Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used as described herein may be made by the hybridoma method first described by Kohler et al. [39], or may be made by recombinant DNA methods (see, e.g., U.S. Pat. No. 4,816,567), or any other suitable methodology known and available in the art. The “monoclonal antibodies” may also be isolated from phage antibody libraries using the techniques described in Clackson et al. [41] and Marks et al. [42], for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity and/or specificity [43-44]. Methods of making chimeric antibodies are known in the art.

An “isolated” antibody is one that has been identified and separated and/or recovered from a component of its natural environment. Contaminant components of its natural environment are materials that would interfere with diagnostic or prognostic uses for the antibody, and may include enzymes, hormones, and other proteinaceous or non-proteinaceous solutes. In specific embodiments, the antibody will be purified to greater than 95% by weight of antibody, e.g., as determined by the Lowry method, and most preferably more than 99% by weight.

The terms “detectably labeled antibody” refers to an antibody (or antibody fragment) which retains binding specificity for a protein marker described herein, and which has an attached detectable label. The detectable label can be attached by any suitable means, e.g., by chemical conjugation or genetic engineering techniques. Methods for production of detectably labeled proteins are well known in the art. Detectable labels may be selected from a variety of such labels known in the art, including, but not limited to, haptens, radioisotopes, fluorophores, paramagnetic labels, enzymes (e.g., horseradish peroxidase), or other moieties or compounds which either emit a detectable signal (e.g., radioactivity, fluorescence, color) or emit a detectable signal after exposure of the label to its substrate. Various detectable label/substrate pairs (e.g., horseradish peroxidase/diaminobenzidine, avidin/streptavidin, and luciferase/luciferin)), methods for labeling antibodies, and methods for using labeled antibodies are well known in the art (see, for example, 45).

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, immunoassays are routinely used to select antibodies specifically immunoreactive with a protein.

An example immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “VII,” or “VH” refer to the variable region of an immunoglobulin heavy chain, including an Fv, scFv, dsFv or Fab, while the terms “variable light chain,” “VL” or “VL” refer to the variable region of an immunoglobulin light chain, including of an Fv, scFv, dsFv or Fab.

“Functional fragments” of antibodies can also be used and include those fragments that retain sufficient binding affinity and specificity for a protein marker to permit a determination of the level of the protein marker in a biological sample. In some cases, a functional fragment will bind to a protein marker with substantially the same affinity and/or specificity as an intact full chain molecule from which it may have been derived. Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen. As appreciated by one of skill in the art, various antibody fragments can be obtained by a variety of methods, for example, digestion of an intact antibody with an enzyme, such as pepsin, or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use as described herein include humanized and/or chimeric monoclonal antibodies.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form.

A “substantially isolated” or “isolated” substance is one that is substantially free of its associated surrounding materials in nature. By substantially free is meant at least 50%, preferably at least 70%, more preferably at least 80%, and even more preferably at least 90% free of the materials with which it is associated in nature. As used herein, “isolated” can refer to polynucleotides, polypeptides, antibodies, cells, samples, and the like.

As used herein, “adiponectin” refers to a protein involved in regulating glucose as well as fatty acid breakdown. It is also referred to as GBP-28, apM1, AdipoQ, and Acrp30. Adiponectin is a 244-amino-acid peptide secreted by adipose tissue, whose roles include the regulation of glucose and fatty acid metabolism.

As used herein, “angiopoietin 1” refers to is a type of angiopoietin and is encoded by the gene ANGPT1. Angiopoietins are proteins with important roles in vascular development and angiogenesis. All angiopoietins bind with similar affinity to an endothelial cell-specific tyrosine-protein kinase receptor. The protein encoded by this gene is a secreted glycoprotein that activates the receptor by inducing its tyrosine phosphorylation. It plays a critical role in mediating reciprocal interactions between the endothelium and surrounding matrix and mesenchyme. The protein also contributes to blood vessel maturation and stability, and may be involved in early development of the heart.

As used herein, “apolipoprotein(a)”, also referred to as “apo(a)”, is the main constituent of lipoprotein(a) (Lp(a)). Apolipoprotein(a) has serine proteinase activity and is capable of auto proteolysis. Apolipoprotein(a) inhibits tissue-type plasminogen activator 1. Apolipoprotein(a) is known to be proteolytically cleaved, leading to the formation of the so-called mini-Lp(a). Apolipoprotein(a) fragments accumulate in atherosclerotic lesions, where they may promote thrombogenesis.

As used herein, “apolipoprotein A-II” refers to an apolipoprotein found in high-density lipoprotein (HDL) cholesterol in plasma. Apolipoprotein (apo) A-II is the second major apo of high-density lipoproteins. Results suggest that enrichment of apo A-II in high-density lipoprotein particles may have athero-protective effects, although its exact mechanism is unclear. Apo A-II may become a target for the treatment of atherosclerosis.

As used herein, “apolipoprotein C-I” is a protein component of lipoproteins normally found in the plasma and responsible for the activation of esterified lecithin cholesterol and in removal of cholesterol from tissues.

As used herein, “angiotensin converting enzyme” or “ACE” refers to a central component of the renin-angiotensin system (RAS), which controls blood pressure by regulating the volume of fluids in the body. It converts the hormone angiotensin Ito the active vasoconstrictor angiotensin II.

As used herein, “blood urea nitrogen” or “BUN” refers to a medical test that measures the amount of urea nitrogen found in blood. The liver produces urea in the urea cycle as a waste product of the digestion of protein.

As used herein, “creatinine” refers to a by-product of everyday muscle contraction while blood urea nitrogen measures the amount of urea nitrogen, a by-product of the urea cycle that breaks down amino acids, in the blood.

As used herein, “blood urea nitrogen to creatinine ratio” or “BCR” is a common laboratory test.

As used herein, “CD5 antigen-like” or “CD5L”, also known as “apoptosis inhibitor of macrophage”, is a protein that is expressed in inflamed tissues.

As used herein, “C reactive protein” or “CRP” is an acute-phase reactant protein that responds rapidly to inflammation.

As used herein, “carcinoembryonic antigen related cell adhesion molecule 1” or “biliary glycoprotein” or “CEACAM1” also known as “CD66a” (Cluster of Differentiation 66a), is a human glycoprotein that mediates cell adhesion via homophilic as well as heterophilic binding to other proteins of the subgroup.

As used herein, “cystatin”, also known as “Cystatin C” or “cystatin 3” (formerly “gamma trace”. “post-gamma-globulin”, or “neuroendocrine basic polypeptide”), is a protein encoded by the CST3 gene, is mainly used as a biomarker of kidney function. Recently, it has been studied for its role in predicting new-onset or deteriorating cardiovascular disease. Cystatin belongs to the type 2 cystatin gene family.

As used herein, “decorin”, also known as “PG40” and “PGS2”, is a protein, which belongs to the small leucine-rich proteoglycan family. It regulates assembly of the extracellular collagen matrix.

As used herein, “eotaxin 1”, also known as “C-C motif chemokine 11” and “eosinophil chemotactic protein” is a small cytokine belonging to the CC chemokine family.

As used herein “ENRAGE”, also known as “extracellular newly identified receptor for advanced glycation end-products binding protein”, has been implicated in various inflammatory diseases and/or states including cardiovascular disease

As used herein “Factor VII”, also known as “blood-coagulation factor VIIa”, “activated blood coagulation factor VII”, or “proconvertin” is one of the proteins that causes blood to clot in the coagulation cascade. It is an enzyme of the serine protease class.

As used herein “ferritin” is a universal intracellular protein that stores iron and releases it in a controlled fashion.

As used herein fetuin A, also known as “alpha-2-HS-glycoprotein” or “AHSG” is a protein that belongs to the fetuin class of plasma binding proteins and is more abundant in fetal than adult blood.

As used herein “follicle stimulating hormone” or “FSH”, is a gonadotropin, a glycoprotein polypeptide hormone. FSH is synthesized and secreted by the gonadotropic cells of the anterior pituitary gland and regulates the development, growth, pubertal maturation, and reproductive processes of the body.

As used herein “growth hormone”, also known as “somatotropin” or as “human growth hormone” or “hGH” in its human form, is a peptide hormone that stimulates growth, cell reproduction, and cell regeneration in humans and other animals. It is thus important in human development. It is a type of mitogen specific only to certain kinds of cells. GH is a stress hormone that raises the concentration of glucose and free fatty acids.

As used herein “immunoglobulin M” or “IgM” is one of several forms of antibody that are produced by vertebrates. IgM is the largest antibody, and it is the first antibody to appear in the response to initial exposure to an antigen.

As used herein “Intercellular Adhesion Molecule 1” also known as “ICAM-1” and “CD54” or “Cluster of Differentiation 54” is a cell surface glycoprotein, which is typically expressed on endothelial cells and cells of the immune system. It binds to integrins of type CD11a/CD18, or CD11b/CD18.

As used herein “interferon gamma induced protein 10”, also known as “CXCL10”, “IP-10” and “10 kDa interferon-gamma-induced protein”, is considered a member of the CXC chemokine and is induced in a variety of cells in response to IFN-gamma. It has proven to be a valid protein marker for the development of cardiovascular disease, including heart failure and left ventricular dysfunction, suggesting an underlining pathophysiological relation with the development of adverse cardiac remodeling.

As used herein, “interleukin-1 receptor antagonist” or “IL-RA” also known as “interleukin 1 inhibitor” or “IL-1 inhibitor”, refers to a protein that is a member of the interleukin 1 cytokine family. IL-RA is secreted by various types of cells including immune cells, epithelial cells, and adipocytes, and is a natural inhibitor of the pro-inflammatory effect of IL1β. This protein inhibits the activities of interleukin 1, alpha (IL1A) and interleukin 1, beta (IL1B), and modulates a variety of interleukin 1 related immune and inflammatory responses.

As used herein, “interleukin-8”, also known as “IL8”, “neutrophil chemotactic factor”, “chemokine ligand 8”, and “CXCL8”, is a chemokine produced by macrophages and other cell types such as epithelial cells, airway smooth muscle cells, and endothelial cells. It induces chemotaxis in target cells, primarily neutrophils but also other granulocytes, causing them to migrate toward the site of infection. IL-8 also induces phagocytosis once they have arrived. IL-8 is also known to be a potent promoter of angiogenesis. In target cells, IL-8 induces a series of physiological responses required for migration and phagocytosis, such as increases in intracellular Ca²⁺, exocytosis (e.g. histamine release), and the respiratory burst.

As used herein, “interleukin-18” also known as “IL-18”, is a proinflammatory cytokine produced by macrophages and other cells. IL-18 works by binding to the interleukin-18 receptor, and together with IL-12, it induces cell-mediated immunity following infection with microbial products like lipopolysaccharide (LPS). After stimulation with IL-18, natural killer (NK) cells and certain T cells release another important cytokine called interferon-γ (IFN-γ) or type II interferon that plays an important role in activating the macrophages or other cells.

As used herein, “interleukin-23”, also known as “IL-23”, is a heterodimeric cytokine composed of an IL12B (IL-12p40) subunit (that is shared with IL12) and the IL23A (IL-23p19) subunit. It has been shown to facilitate development of inflammation in numerous other models of immune pathology where IL-12 had previously been implicated including models of arthritis, intestinal inflammation and psoriasis.

As used herein, “kidney injury molecule 1”, also known as “kidney injury molecule-1” and “KIM-1” is a type I cell membrane glycoprotein that serves as a receptor for oxidized lipoproteins and plays a functional role in the kidney. KIM-1 is a proximal renal tubular marker, concentrations of which have been linked to acute kidney injury.

As used herein, “lipoprotein(a)”, also known as “Lp(a)”, is a subclass of lipoproteins. It a consists of an LDL-like particle and the specific apolipoprotein(a) (apo(a)), which is covalently bound to the apolipoprotein B of the LDL like particle. Lp(a) as a risk factor for atherosclerotic cardiovascular diseases.

As used herein, “matrix metalloproteinase 7” also known as “MMP-7”, “Matrilysin”, “pump-1 protease (PUMP-11”, or “uterine metalloproteinase”, is an enzyme with a primary role to break down extracellular matrix.

As used herein, “matrix metalloproteinase 9”, also known as “MMP-9”, “92 kDa type IV collagenase”, “92 kDa gelatinase”, and “gelatinase B” or “GELB”, is a matrixin, a class of enzymes that belong to the zinc-metalloproteinase family involved in the degradation of the extracellular matrix. Proteins of the matrix metalloproteinase (MMP) family are involved in the breakdown of extracellular matrix in normal physiological processes, such as embryonic development, reproduction, angiogenesis, bone development, wound healing, cell migration, learning and memory, as well as in pathological processes, such as arthritis, intracerebral hemorrhage, and metastasis.

As used herein, “matrix metalloproteinase 9 Total”, also known as “MMP-9 Total”, refers to a combination and/or ratio of matrix metalloproteinase 9 (MMP9) and tissue inhibitor of metalloproteinase 1 (TIMP-1). Matrix metalloproteinase 9, also known as MMP-9, 92 kDa type IV collagenase, 92-kDa gelatinase, and gelatinase B or GELB, is a matrixin, a class of enzymes that belong to the zinc-metalloproteinase family involved in the degradation of the extracellular matrix. Proteins of the matrix metalloproteinase (MMP) family are involved in the breakdown of extracellular matrix in normal physiological processes, such as embryonic development, reproduction, angiogenesis, bone development, wound healing, cell migration, learning and memory, as well as in pathological processes, such as arthritis, intracerebral hemorrhage, and metastasis. TIMP-1 is a glycoprotein that is expressed from several tissues. It is a member of the TIMP family and is a natural inhibitor of the matrix metalloproteinases (MMPs), a group of peptidases involved in degradation of the extracellular matrix. In addition to its inhibitory role against most of the known MMPs, the encoded protein is able to promote cell proliferation in a wide range of cell types, and may also have an anti-apoptotic function. TIMP-1 has been associated plaque rupture and adverse cardiovascular events.

As used herein, “midkine”, also known as “neurite growth-promoting factor 2” or “NEGF2”, refers to a basic heparin-binding growth factor of low molecular weight and forms a family with pleiotrophin. Midkine is a heparin-binding cytokine/growth factor with a molecular weight of 13 kDa.

As used herein, “monokine induced by gamma interferon”, also known as “MIG” or “CXCL9”, is a small cytokine belonging to the family of CXC chemokines. It is a T-cell chemoattractant and has been associated with worsening left ventricular dysfunction and symptomatic cardiovascular disease.

As used herein, “myeloid progenitor inhibitory factor 1” also known as Chemokine (C-C motif) ligand 23, “CCL23”, “Macrophage inflammatory protein 3”, and “MIP-3” is a small cytokine belonging to the CC chemokine family. It is predominantly expressed in lung and liver tissue, but is also found in bone marrow and placenta. It is also expressed in some cell lines of myeloid origin.

As used herein, “myeloperoxidase” also known as “MPO” is a white blood cell-derived inflammatory enzyme that measures disease activity from the luminal aspect of the arterial wall. When the artery wall is damaged, or inflamed, myeloperoxidase is released by invading macrophages where it accumulates. Myeloperoxidase mediates the vascular inflammation that propagates plaque formation and activates protease cascades that are linked to plaque vulnerability.

As used herein, “myoglobin”, is an iron- and oxygen-binding protein found in the muscle tissue of vertebrates in general and in almost all mammals. Myoglobin is released from damaged muscle tissue (rhabdomyolysis), which has very high concentrations of myoglobin. The released myoglobin is filtered by the kidneys but is toxic to the renal tubular epithelium and so may cause acute kidney injury. It is not the myoglobin itself that is toxic (it is a protoxin) but the ferrihemate portion that is dissociated from myoglobin in acidic environments (e.g., acidic urine, lysosomes). Myoglobin is a sensitive marker for muscle injury, making it a potential marker for heart attack in patients with chest pain.

As used herein, “N-terminal prohormone of brain natriuretic peptide” or “NT-PBNP” is also known as “NT-proBNP” or “BNPT” and refers to an N-terminal inactive protein that is cleaved from proBNP to release brain natriuretic peptide.

As used herein, “osteopontin”, also known as “bone sialoprotein I”, “BSP-1”, “BNSP”, “early T-lymphocyte activation”, “ETA-1”, “secreted phosphoprotein 1”, “SPP1”, “2ar”, “Rickettsia resistance”, or “Ric”, refers to a glycoprotein (small integrin binding ligand N-linked glycoprotein) first identified in osteoblasts. It includes all isoforms and post-translational modifications.

As used herein, “pulmonary surfactant associated protein D”, also referred to as surfactant, pulmonary-associated protein D, or SP-D or SFTPD, is a protein that contributes to the lung's defense against inhaled microorganisms, organic antigens and toxins.

As used herein, “resistin” also known as “adipose tissue-specific secretory” factor or “ADSF” or “C/EBP-epsilon-regulated myeloid-specific secreted cysteine-rich protein” or “XCP1” is a cysteine-rich adipose-derived peptide hormone. Resistin increases the production of LDL in human liver cells and degrades LDL receptors in the liver. As a result, the liver is less able to clear ‘bad’ cholesterol from the body. Resistin accelerates the accumulation of LDL in arteries, increasing the risk of heart disease. Resistin adversely impacts the effects of statins, the main cholesterol-reducing drug used in the treatment and prevention of cardiovascular disease.

As used herein, “serotransferrin”, also known as “transferrin”, is an abundant blood plasma glycoprotein with a main function of binding and transporting iron throughout the body. In patients with cardiovascular disease, low levels of serotransferrin causes iron deficiency, which correlates with decreased exercise capacity and poor quality of life, and predicts worse outcomes.

As used herein, “stem cell factor”, also known as SCF, KIT-ligand, KL, and steel factor, is a cytokine that binds to the c-KIT receptor (CD117). SCF can exist as both a transmembrane protein and a soluble protein. This cytokine plays an important role in hematopoiesis (formation of blood cells), spermatogenesis, and melanogenesis.

As used herein, “Tamm Horsfall Urinary Glycoprotein” or “THP”, also known as “uromodulin”, is a glycoprotein that is the most abundant protein excreted in ordinary urine.

As used herein, “tissue inhibitor of” metalloproteinase 1, also known as “TIMP-1” or “TIMP metallopeptidase inhibitor 1”, is a glycoprotein expressed in several tissues. It is a member of the TIMP family and is a natural inhibitor of the matrix metalloproteinases (MMPs), a group of peptidases involved in degradation of the extracellular matrix. In addition to its inhibitory role against most of the known MMPs, the encoded protein is able to promote cell proliferation in a wide range of cell types, and may have an anti-apoptotic function. TIMP-1 has been associated plaque rupture and adverse cardiovascular events.

As used herein, “T Cell Specific Protein RANTES”, also known as “RANTES”, “regulated on activation, normal T cell expressed and secreted”, “Chemokine (C-C motif) ligand 5”, or “CCLS”, is a protein that is chemotactic for T cells, eosinophils, and basophils, and plays an active role in recruiting leukocytes into inflammatory sites. With the help of particular cytokines (i.e., IL-2 and IFN-y) that are released by T cells, CCLS also induces the proliferation and activation of certain natural-killer (NK) cells to form CHAK (CC-Chemokine-activated killer) cells.

As used herein, “Thyroxine Binding Globulin”, or “TBG” a globulin that binds thyroid hormones in circulation. It is one of three transport proteins (along with transthyretin and serum albumin) responsible for carrying the thyroid hormones thyroxine (T4) and triiodothyronine (T3) in the bloodstream.

As used herein, “transthyretin” or “TTR” is a transport protein in the serum and cerebrospinal fluid that carries the thyroid hormone thyroxine (T₄) and retinol-binding protein bound to retinol.

As used herein, “troponin”, also known as the troponin complex, is a complex of three regulatory proteins (troponin C, troponin I, and troponin T) that is integral to muscle contraction in skeletal muscle and cardiac muscle, but not smooth muscle. As used herein a troponin protein marker may identify each of these proteins individually, or in combination, and may be any level of sensitivity. An increased level of the cardiac protein isoform of troponin circulating in the blood has been shown to be a protein marker of heart disorders and heart stress, the most important of which is myocardial infarction. Raised troponin levels indicate cardiac muscle cell death or damage as the molecule is released into the blood upon injury to the heart.

As used herein, “vascular cell adhesion molecule”, also known as VCAM-1, VCAM, cluster of differentiation 106, and CD106, is a cell adhesion molecule. The VCAM-1 protein mediates the adhesion of lymphocytes, monocytes, eosinophils, and basophils to vascular endothelium. It also functions in leukocyte-endothelial cell signal transduction, and it may play a role in the development of atherosclerosis and rheumatoid arthritis.

As used herein, “vitamin D binding protein”, also known as “gc-globulin” or “group-specific component”, belongs to the albumin gene family, together with human serum albumin and alpha-fetoprotein. It is a multifunctional protein found in plasma, ascetic fluid, and cerebrospinal fluid and on the surface of many cell types. It is able to bind the various forms of vitamin D including ergocalciferol (vitamin D₂) and cholecalciferol (vitamin D₃), the 25-hydroxylated forms (calcifediol), and the active hormonal product, 1,25-dihydroxyvitamin D (calcitriol). The major proportion of vitamin D in blood is bound to this protein. It transports vitamin D metabolites between skin, liver and kidney, and then on to the various target tissues.

As used herein, “von Willebrand Factor” or “vWF” is a blood glycoprotein involved in hemostasis. Its primary function is binding to other proteins, in particular factor VIII, cells, and molecules. It is important in platelet adhesion to wound sites, thus playing a major role in blood coagulation. It is not an enzyme and, thus, has no catalytic activity.

It will be understood by one skilled in the art that these and other protein markers disclosed herein (e.g., those set forth in Table 1) can be readily identified, made and used in the context of the present disclosure in light of the information provided herein.

As used herein, the term “score” refers to a binary, multilevel, or continuous result as it relates diagnostic or prognostic determinations. A score can be a positive, intermediate, or negative diagnostic score. A score can be a positive, intermediate, or negative prognostic score. One or multiple cutoffs can be used with the score to determine specific levels of risk. In embodiments, a score is algorithmically derived based on normalized and/or mathematically transformed values, such as protein concentrations, the presence/absence of clinical factors, vital statistics, or ratios of different factors. The algorithm which generates the score can be ratio-based, cut-off-based, linear or non-linear, including decision tree or rule-based models.

As used herein, the term “panel” refers to specific combination of protein markers and clinical markers used to determine a diagnosis or prognosis of a cardiovascular disease or outcome in a subject. The term “panel” may also refer to an assay comprising a set of protein markers used to determine a diagnosis or prognosis of a cardiovascular disease or outcome in a subject.

As further described herein, the “training set” is the set of patients or patient samples that are used in the process of training (i.e., developing, evaluating and building) the final diagnostic or prognostic model. The “validation set” is a set of patients or patient samples that are withheld from the training process, and are only used to validate the performance of the final diagnostic or prognostic model. If the set of patients or patient samples are limited in number, all available data may be used as a training set, or as an “in-sample” validation set.

As used herein, the term “normalized” refers to a type of transformation where the values are designed to fit a specific distribution, typically so that they are similar to the distributions of other variables. For example, for hypothetical proteins A and B, the raw concentration of protein A ranges from 0 to 500 and the raw concentration of Protein B ranges from 0 to 20,000, it is not trivial looking at thee raw values to determine which one is “higher”. For instance, is 400 of Protein A higher than 15,000 of Protein B. By conducting a normalization process, the concentrations are rescaled so that they are on the same scale: centered at zero, with a variance of 1. Thus, it becomes a routine exercise to determine which one is higher because the normalized concentrations are comparable. Many learning algorithms work better on data that are normalized; otherwise, in this example for instance, Protein B might get more weight in the algorithm because it has higher values even if it were not empirically “higher.”

As used herein, the term “transformed” refers to a mathematical process applied to a numerical value, regardless of the input or output value. It may include taking protein concentrations and calculating the base-10 logarithm from original values, reflecting a “log-transformation.”

### PROTEIN MARKERS

Certain illustrative protein markers provided herein can be found listed in Table 1. Based on the information therein, the skilled artisan can readily identify, select and implement a protein marker or protein marker combination in accordance with the methods provided herein.

In embodiments, at least 2, at least 3 or at least 4 protein markers from Table 1 are used in the methods and panels provided herein. In an embodiment, two proteins from Table 1 are selected. In an embodiment, three proteins from Table 1 are selected. In an embodiment, four proteins from Table 1 are selected. In an embodiment, five proteins from Table 1 are selected. In an embodiment, six proteins from Table 1 are selected. In other embodiments, the number of protein markers employed can vary, and may include at least 5, 6, 7, 8, 9, 10, or more. In still other embodiments, the number of protein markers can include at least 15, 20, 25 or 50, or more. Also, in some embodiments, one or more of the protein markers from Table 1 can be specifically excluded. For example, 1, 2, 3, 4, 5, 6, 7 or more of the specific protein markers can be excluded from some embodiments, in any combination.

In certain specific embodiments, the protein markers used herein include those listed in Table 1, particularly those that are associated with a p-value of less than 0.1, less than 0.05, less than 0.01 or less than 0.001.

In embodiments, the protein markers used in herein are selected from adiponectin, angiopoietin 1, apolipoprotein(a), apolipoprotein C-I, angiotensin converting enzyme, carcinoembryonic antigen related cell adhesion molecule 1, eotaxin 1, ENRAGE, Factor VII, ferritin, fetuin A, follicle stimulating hormone, growth hormone, immunoglobulin M, intercellular adhesion molecule 1, interferon gamma induced protein 10, interleukin 1 receptor antagonist, interleukin 8, interleukin 18, interleukin 23, kidney injury molecule 1, matrix metalloproteinase 7, matrix metalloproteinase 9 Total, midkine, monokine induced by gamma interferon, myeloid progenitor inhibitory factor 1, N terminal prohormone of brain natriuretic peptide, osteopontin, pulmonary surfactant associated protein D, resistin, serotransferrin, Tamm Horsfall urinary glycoprotein, T Cell specific protein RANTES, thyroxine binding globulin, transthyretin, vitamin D binding protein, and von Willebrand factor. In some embodiments, one or more (any combination) of the above-listed protein markers can be specifically excluded from any of the embodiments and aspects described herein.

In embodiments, the protein markers used in accordance with the present disclosure are selected from angiopoietin 1, apolipoprotein C-I, angiotensin converting enzyme, carcinoembryonic antigen related cell adhesion molecule 1, eotaxin 1, ENRAGE, fetuin A, follicle stimulating hormone, intercellular adhesion molecule 1, interferon gamma induced protein 10, interleukin 1 receptor antagonist, interleukin 8, interleukin 23, kidney injury molecule 1, matrix metalloproteinase 7, matrix metalloproteinase 9 Total, midkine, monokine induced by gamma interferon, myeloid progenitor inhibitory factor 1, osteopontin, pulmonary surfactant associated protein D, resistin, serotransferrin, Tamm Horsfall urinary glycoprotein, T cell specific protein RANTES, thyroxine binding globulin, and transthyretin. In some embodiments, one or more (any combination) of the above-listed protein markers can be specifically excluded from any of the embodiments and aspects described herein.

In still other embodiments, the protein markers used in accordance with the present disclosure are selected from factor VII, ferritin, growth hormone, immunoglobulin M, kidney injury molecule 1, and vitamin D binding protein. In some embodiments, one or more (any combination) of the above-listed protein markers can be specifically excluded from any of the embodiments and aspects described herein.

In still other embodiments, the protein markers used in accordance with the present disclosure are selected from adiponectin, apolipoprotein(a), fetuin A, interleukin 18, N terminal prohormone of brain natriuretic peptide, osteopontin, resistin, and von Willebrand factor. In some embodiments, one or more (any combination) of the above-listed protein markers can be specifically excluded from any of the embodiments and aspects described herein.

In embodiments, as noted elsewhere herein, a protein as recited in Table 1 may be specifically excluded from the methods or panels described herein.

TABLE 1
is a list of 113 proteins whose levels are correlated to the diagnosis,
monitoring, and/or prognosis of a cardiovascular disease or event,
specifically peripheral artery disease, limb amputation, and aortic
stenosis.
Adiponectin
Alpha 1 Antitrypsin
Alpha 2 Macroglobulin
Angiopoietin 1
Angiotensin Converting Enzyme
Apolipoprotein(a)
Apolipoprotein AI
Apolipoprotein AII
Apolipoprotein B
Apolipoprotein CI
Apolipoprotein CIII
Apolipoprotein H
Beta 2 Microglobulin
Brain Derived Neurotrophic Factor
C Reactive Protein
Calbindin
Carbonic anhydrase 9
Carcinoembryonic antigen related cell adhesion molecule 1
CD5 Antigen like
Cystatin
Decorin
E Selectin
ENRAGE
Eotaxin 1
Factor VII
Fatty Acid Binding Protein
Ferritin
Fetuin A
Fibrinogen
Follicle Stimulating Hormone
Glucagon-like Peptide-1
Granulocyte Macrophage Colony Stimulating Factor
Growth Hormone
Haptoglobin
Immunoglobulin A
Immunoglobulin M
Insulin
Intercellular Adhesion Molecule-1
Interferon gamma
Interferon-gamma-Induced-Protein 10
Interleukin-1 alpha
Interleukin-1 beta
Interleukin-1 receptor antagonist
Interleukin-2
Interleukin-3
Interleukin-4
Interleukin-5
Interleukin-6
Interleukin-6 receptor
Interleukin-7
Interleukin-8
Interleukin-10
Interleukin-12 Subunit p40
Interleukin-12 Subunit p70
Interleukin-15
Interleukin-17
Interleukin-18
Interleukin-18 binding protein
Interleukin-23
Kidney Injury Molecule 1
Lectin Like Oxidized LDL Receptor 1
Leptin
Lipoprotein(a) (Lp(a))
Luteinizing Hormone
Macrophage Colony Stimulating Factor 1
Macrophage Inflammatory Protein 1 alpha
Macrophage Inflammatory Protein 1 beta
Macrophage Inflammatory Protein 3 alpha
Matrix Metalloproteinase 1
Matrix Metalloproteinase 2
Matrix Metalloproteinase 3
Matrix Metalloproteinase 7
Matrix Metalloproteinase 9
Matrix Metalloproteinase 9 Total
Matrix Metalloproteinase 10
Midkine
Monocyte Chemotactic Protein 1
Monocyte Chemotactic Protein 2
Monocyte Chemotactic Protein 4
Monokine Induced by Gamma Interferon
Myeloid Progenitor Inhibitory Factor 1
Myeloperoxidase
Myoglobin
N terminal prohormone of brain natriuretic peptide
Osteopontin
Pancreatic Polypeptide
Plasminogen Activator Inhibitor 1
Platelet endothelial cell adhesion molecule
Prolactin
Pulmonary and Activation Regulated Chemokine
Pulmonary surfactant-associated protein D
Resistin
Serotransferrin
Serum Amyloid P Component
Stem Cell Factor
T-Cell-Specific Protein RANTES
Tamm Horsfall Urinary Glycoprotein
Thrombomodulin
Thrombospondin 1
Thyroid Stimulating Hormone
Thyroxine Binding Globulin
Tissue Inhibitor of Metalloproteinases 1 (TIMP-1)
Transthyretin
Troponin
Tumor Necrosis Factor alpha
Tumor Necrosis Factor beta
Tumor necrosis factor receptor 2
Vascular Cell Adhesion Molecule 1
Vascular Endothelial Growth Factor
Vitamin D Binding Protein
Vitamin K-Dependent Protein S
Vitronectin
von Willebrand Factor

In embodiments, the combination of proteins whose concentrations are correlated to the diagnosis, monitoring, and/or prognosis of a cardiovascular disease or event, specifically peripheral artery disease, limb amputation, and aortic stenosis and the nature of whether those protein concentrations are increased, decreased, or the same as compared to a healthy individual provides a subject's protein profile.

### CLINICAL VARIABLES

As further described herein, the protein markers described herein can optionally be used in combination with certain clinical variables in order to provide for an improved diagnosis and/or prognosis of a cardiovascular disease or event in a subject. As used herein, “optionally” refers to inclusion based on combinations of protein markers and their predictive value of cardiovascular disease or outcome when combined with a clinical variable factor. For example, illustrative clinical variables useful in the context of the present disclosure can be found listed in Table 2.

In embodiments, at least 1, at least 2, at least 3 or at least 4 clinical variables from Table 2 are used in the methods and panels provided herein. In an embodiment, one clinical variable from Table 2 is selected. In an embodiment, two clinical variables from Table 2 are selected. In an embodiment, three clinical variables from Table 2 are selected. In an embodiment, four clinical variables from Table 2 are selected. In an embodiment, five clinical variables from Table 2 are selected. In other embodiments, the number of clinical variables employed can vary, and may include at least 5, 6, 7, 8, 9, 10, or more.

In embodiments, the clinical variable(s) used in accordance with the present disclosure are selected from age, history of hypertension, history of diabetes mellitus Type 2, smoker, history of dyslipidemia, body mass index (BMI), history of peripheral percutaneous angioplasty (with or without stent), history of peripheral revascularization, and history of coronary revascularization. In embodiments, the clinical variable used in accordance with the present disclosure is age. In embodiments, the clinical variable used in accordance with the present disclosure is history of hypertension. In embodiments the clinical variable used in accordance with the present disclosure is history of percutaneous peripheral angioplasty (with or without stent). In embodiments, the clinical variable used in accordance with the present disclosure is history of peripheral revascularization intervention (peripheral angioplasty, stent or bypass). In embodiments, the clinical variable used in accordance with the present disclosure is diabetes mellitus type 2. In embodiments, the clinical variables used in accordance with the present disclosure are history of peripheral revascularization intervention (peripheral angioplasty, stent or bypass) and history of hypertension. In embodiments, the clinical variables used in accordance with the present disclosure are body mass index and history of hypertension. In embodiments, the clinical variables used in accordance with the present disclosure are history of dyslipidemia and history of hypertension. In embodiments, the clinical variables used in accordance with the present disclosure are history of diabetes type 2 and smoker. In embodiments, the clinical variables used in accordance with the present disclosure are age and history of coronary revascularization intervention (coronary angioplasty, stent or bypass). In some embodiments, one or more (any combination) of the above-listed clinical variables can be specifically excluded from any of the embodiments and aspects described herein.

In embodiments, the presence/absence of clinical factors represented in binary form (e.g., sex), and/or clinical factors in quantitative form (e.g., BMI, age) provide values that are entered into the diagnostic model provided by the software, and the result is evaluated against one or more cutoffs to determine the diagnosis or prognosis.

In embodiments, one or more (any combination) of the clinical characteristic as recited in Table 2 may be specifically excluded from the methods and other embodiments described herein.

TABLE 2
is a list of clinical variables and lab measurements correlated to the
diagnosis or prognosis of a cardiovascular disease or event, specifically
peripheral artery disease, limb amputation, and aortic stenosis.
Clinical Characteristics
Demographics
Age
Sex
Race
Vital Signs
Body Mass Index (BMI)
Heart rate (beat/min)
Systolic BP (mmHg)
Diastolic BP (mmHg)
Medical History
Current smoker
Former smoker
History of atrial fibrillation/flutter
History of dyslipidemia
History of hypertension
History of coronary artery disease (CAD)
History of myocardial infarction (MI)
History of heart failure (HF)
History of peripheral artery disease (PAD)
History of COPD
History of diabetes mellitus, Type 1
History of diabetes mellitus, Type 2
History of any Diabetes
History of CVA/TIA
History of chronic kidney disease (CKD)
History of hemodialysis
History of angioplasty, (peripheral and/or coronary)
History of stent (peripheral and/or coronary)
History of CABG
History of coronary revascularization intervention (coronary angioplasty,
stent or bypass)
History of percutaneous coronary intervention
History of peripheral revascularization
History of percutaneous peripheral intervention
History of percutaneous peripheral angioplasty (with or without stent)
History of resuscitation from sudden cardiac death
Family history of CAD
History of significant ventricular arrhythmia or suspected SCD (not in the
setting of acute MI)
Medications
ACE-I/ARB
Beta blocker
Aldosterone antagonist
Loop diuretics
Nitrates
CCB
Statin
Aspirin
Warfarin
Clopidogrel
Echocardiographic results
LVEF (%)
RSVP (mmHg)
Aortic valve area (AVA) (cm²)
Left ventricular internal diameter in end diastole (cm)
Posterior wall thickness of left ventricle (mm)
Inter-ventricular septal wall thickness (mm)
Left ventricular mass (grams)
Relative wall thickness (ratio of twice left ventricular diastolic wall
thickness to left ventricular end-diastolic dimension)
Mitral regurgitation (none, trace, mild, moderate, severe)
Aortic regurgitation (none, trace, mild, moderate, severe)
Tricuspid regurgitation (none, trace, mild, moderate, severe)
Peak velocity across aortic valve (cm/sec)
Left ventricular outflow tract velocity (cm/sec)
Peak gradient across aortic valve (mmHg)
Mean gradient across aortic valve (mmHg)
Stress test results
Ischemia on Scan
Ischemia on ECG
Angiography results
≥70% coronary stenosis
≥50% stenosis in at least one peripheral vessel
Lab Measures
Sodium
Blood urea nitrogen (mg/dL)
Creatinine (mg/dL)
Blood Urea Nitrogen: Creatinine Ratio
eGFR (median, CKDEPI)
Total cholesterol (mg/dL)
LDL cholesterol (mg/dL)
Glycohemoglobin (%)
Glucose (mg/dL)
HGB (mg/dL)
BP = blood pressure,
CAD = coronary artery disease,
MI = myocardial infarction,
HF = heart failure,
COPD = chronic obstructive pulmonary disease,
CVA/TIA = cerebrovascular accident/transient ischemic attack,
CKD = chronic kidney disease,
SCD = sudden cardiac death,
CABG = coronary artery bypass graft,
ACE-I/ARB = angiotensin converting enzyme inhibitor/angiotensin receptor blocker,
CCB = calcium channel blocker,
LVEF = left ventricular ejection fraction,
RVSP = right ventricular systolic pressure,
ECG = echocardiogram,
CKDEPI = Chronic Kidney Disease Epidemiology group (a standard for calculating eGFR),
eGFR = estimated glomerular filtration rate,
LDL = low density lipoprotein,
HGB = hemoglobin.

Peripheral Artery Disease

In an aspect, provided herein are methods of determining peripheral artery disease in a subject. The methods include providing a biological sample from a subject suspected of having peripheral artery disease, applying the biological sample to an analytical device that is programmed to detect the concentration of at least two protein markers in the sample, calculate the concentrations against a quantification standard, and transform the normalized concentrations, and calculate a score using an algorithm. The methods include optionally determining the status of at least one clinical variable, calculating a diagnostic score using an algorithm based on the transformed, normalized concentrations of protein markers and optionally, the status of the clinical variable(s), classifying the diagnostic score as a positive, intermediate, or negative result, and determining peripheral artery disease in the subject as indicated by the diagnostic score. The at least two protein markers are selected from Table 1. The optional clinical variable(s) are selected from Table 2.

In an aspect, provided herein are methods of administering a therapeutic intervention to a subject suspected of having peripheral artery disease. The methods include (i) determining the subject's protein marker profile for a panel of at least two protein markers selected from Table 1; (ii) optionally, determining the status of at least one clinical variable for the subject, where the clinical variable is selected from Table 2; (iii) assigning a score to the subject based on the protein marker profile in (i) and optionally the clinical value status in (ii); and (iv) administering to the subject a therapeutic intervention based on the positive, intermediate or negative score. Provided in the methods herein, the score is selected from positive, intermediate, and negative, and the score is algorithmically-derived based on the normalized and mathematically transformed concentrations of protein markers in the subject's sample and optionally, the status of at least one clinical variable.

In an aspect, provided herein are methods of detecting two or more protein markers in a subject having hypertension and/or that is suspected of having peripheral artery disease. The methods include selecting a subject that has hypertension and/or that is suspected of having peripheral artery disease, providing a biological sample from the subject, applying the biological sample to an analytical device, and detecting the concentration of at least two protein markers selected from Table 1.

In embodiments, a positive score indicates strong likelihood or presence of peripheral artery disease. In embodiments, a positive score indicates strong likelihood or presence of >50% obstruction in peripheral arteries. In embodiments, an intermediate score indicates a possible presence or likelihood of peripheral artery disease. In embodiments, an intermediate score indicates a possible presence or likelihood of >50% obstruction in peripheral arteries. In embodiments, a negative score indicates absence or a weak likelihood of peripheral artery disease. In embodiments, a negative score indicates absence or a weak likelihood of >50% obstruction in peripheral arteries.

Peripheral Limb Amputation Risk

In an aspect, provided herein are methods of determining risk of peripheral limb amputation in a subject within a time point. The methods include providing a biological sample from a subject suspected of having a risk of peripheral limb amputation, applying the biological sample to an analytical device that is programmed to detect the concentration of at least two protein markers in the sample. The concentrations are normalized against a quantification standard, and mathematically transformed. The methods include optionally determining the status of at least one clinical variable, calculating a prognostic score using an algorithm based on the normalized, transformed concentrations of protein markers and optionally, the status of the clinical variable(s), classifying the prognostic score as a positive, intermediate, or negative result, and determining the risk of peripheral limb amputation in the subject as indicated by the prognostic score. The at least two protein markers are selected from Table 1. The optional clinical variable is selected from Table 2.

In an aspect, provided herein are methods of administering a therapeutic intervention to a subject suspected of having a risk of peripheral limb amputation. The methods include (i) determining the subject's protein marker profile for a panel of at least two protein markers selected from Table 1; (ii) optionally, determining the status of at least one clinical variable for the subject, where the clinical variable is selected from Table 2; (iii) assigning a score to the subject based on the protein marker profile in (i) and optionally the clinical value status in (i); and (ii) administering to the subject a therapeutic intervention based on the positive, intermediate or negative score. Provided in the methods herein, the score is selected from positive, intermediate, and negative, and the score is algorithmically-derived based on the normalized, mathematically transformed concentrations of protein markers in the subject's sample and optionally, the status of at least one clinical variable.

In an aspect, provided herein are methods of detecting two or more protein markers in a subject having diabetes mellitus type 2 and/or that is suspected of having a risk of peripheral limb amputation. The methods include selecting a subject that has diabetes mellitus type 2 and/or that is suspected of having a risk of peripheral limb amputation, providing a biological sample from the subject, applying the biological sample to an analytical device, and detecting the concentration of at least two protein markers selected from Table 1.

In embodiments, a positive score indicates strong likelihood of a risk for peripheral limb amputation. In embodiments, an intermediate score indicates a possible likelihood of a risk for peripheral limb amputation. In embodiments, a negative score indicates absence or a weak likelihood of a risk for peripheral limb amputation.

Aortic Valve Stenosis

In an aspect, provided herein are methods of determining aortic valve stenosis in a subject. The methods include providing a biological sample from a subject suspected of having aortic stenosis, applying the biological sample to an analytical device that is programmed to detect the concentration of at least two protein markers in the sample, normalize the concentrations against a quantification standard, and transform the normalized concentrations. The methods include optionally determining the status of at least one clinical variable, calculating a diagnostic score using an algorithm based on the normalized, transformed concentrations of protein markers and optionally, the status of the clinical variable(s), classifying the diagnostic score as a positive, intermediate, or negative result, and determining aortic stenosis in the subject as indicated by the diagnostic score. The at least two protein markers are selected from Table 1. The optional clinical variable is selected from Table 2.

In an aspect, provided herein are methods of administering a therapeutic intervention to a subject suspected of having aortic stenosis. The methods include (i) determining the subject's protein marker profile for a panel of at least two protein markers selected from Table 1; (ii) optionally, determining the status of at least one clinical variable for the subject, where the clinical variable is selected from Table 2; (iii) assigning a score to the subject based on the protein marker profile in (i) and optionally the clinical value status in (ii); and (iv) administering to the subject a therapeutic intervention based on the positive, intermediate or negative score. Provided in the methods herein, the score is selected from positive, intermediate, and negative, and the score is algorithmically-derived based on the normalized, mathematically transformed concentrations of protein markers in the subject's sample and optionally, the status of at least one clinical variable.

In embodiments, a positive score indicates strong likelihood or presence of aortic stenosis. In embodiments, an intermediate score indicates a possible presence or likelihood of aortic stenosis. In embodiments, a negative score indicates absence or a weak likelihood of aortic stenosis.

### Embodiments

In certain specific embodiments, protein markers, optionally used in conjunction with clinical variables, can be used in methods for the diagnosis of peripheral artery disease, and/or the prognosis of peripheral intervention and/or monitoring PAD progression or therapeutic effect and/or prognosis for peripheral limb amputation risk. In some embodiments, the protein markers are selected from angiopoietin 1, apolipoprotein C-I, angiotensin converting enzyme, carcinoembryonic antigen related cell adhesion molecule 1, eotaxin 1, ENRAGE, fetuin A, follicle stimulating hormone, intercellular adhesion molecule 1, interferon gamma induced protein 10, interleukin 1 receptor antagonist, interleukin 8, interleukin 23, kidney injury molecule 1, matrix metalloproteinase 7, matrix metalloproteinase 9 Total, midkine, monokine induced by gamma interferon, myeloid progenitor inhibitory factor 1, osteopontin, pulmonary surfactant associated protein D, resistin, serotransferrin, Tamm Horsfall urinary glycoprotein, T cell specific protein RANTES, thyroxine binding globulin, and transthyretin. In embodiments, the methods include determining the clinical variable of age, history of hypertension, history of peripheral percutaneous angioplasty (with or without stent), body mass index (BMI), history of dyslipidemia, and/or history of peripheral revascularization (peripheral angioplasty, stent or bypass). In some embodiments, the protein markers are angiopoietin 1, eotaxin 1, follicle stimulating hormone, interleukin 23, kidney injury molecule 1, midkine and the clinical variable includes history of hypertension. In some embodiments, one or more (any combination) of the above-listed protein markers can be specifically excluded from any of the embodiments and aspects described herein.

In certain specific embodiments, protein markers, optionally used in conjunction with clinical variables, can be used in the prognosis of cardiovascular outcomes, including but not limited to risk of limb amputation. In some embodiments, the protein markers are selected from factor VII, ferritin, growth hormone, immunoglobulin M, kidney injury molecule 1, and vitamin D binding protein. In embodiments, the methods include determining the clinical variable of history of diabetes mellitus type 2 and/or smoker. In some embodiments, the protein markers are kidney injury molecule-1 and vitamin D binding protein and the clinical variable includes determining the status of history of diabetes mellitus type 2. In some embodiments, one or more (any combination) of the above-listed variables can be specifically excluded from any of the embodiments and aspects described herein.

In certain specific embodiments, protein markers, optionally used in conjunction with clinical variables, can be used in the methods described herein for the diagnosis of aortic valve stenosis. In some embodiments, the protein markers are selected from adiponectin, Apolipoprotein(a), fetuin A, interleukin 18, N terminal prohormone of brain natriuretic peptide, osteopontin, resistin, and von Willebrand factor. In embodiments, the methods include determining the clinical variable of age and/or history of coronary revascularization intervention (coronary angioplasty, stent or bypass). In some embodiments, the protein markers are fetuin A, N terminal prohormone of brain natriuretic peptide, and von Willebrand factor and the clinical variable includes determining age.

Assay

In embodiments, the biological sample includes whole blood, plasma, serum, urine, cerebral spinal fluid, biological fluid, and/or tissue samples. In some embodiments, the sample is whole blood. In some embodiments, the sample is plasma. In other embodiments, the sample is serum or urine.

Determining protein marker concentrations in a sample taken from a subject can be accomplished according to standard techniques known and available to the skilled artisan. In many instances, this will involve carrying out protein detection methods, which provide a quantitative measure of protein markers present in a biological sample.

In embodiments, target-binding agents that specifically bind to the protein markers described herein allow for a determination of the concentrations of the protein markers in a biological sample. Any of a variety of binding agents may be used including, for example, antibodies, polypeptides, sugars, aptamers, and nucleic acids.

In embodiments, the binding agent is an antibody or a fragment thereof that specifically binds to a protein marker as provided herein, and that is effective to determine the concentration of the protein marker to which it binds in a biological sample.

The term “specifically binds” or “binds specifically,” in the context of binding interactions between two molecules, refers to high avidity and/or high affinity binding of an antibody (or other binding agent) to a specific polypeptide subsequence or epitope of a protein marker. Antibody binding to an epitope on a specific protein marker sequence (also referred to herein as “an epitope”) is preferably stronger than binding of the same antibody to any other epitope, particularly those that may be present in molecules in association with, or in the same sample, as the specific protein marker of interest. Antibodies which bind specifically to a protein marker of interest may be capable of binding other polypeptides at a weak, yet detectable, level (e.g., 10% or less, 5% or less, 1% or less of the binding shown to the polypeptide of interest). Such weak binding, or background binding, is readily discernible from the specific antibody binding to the compound or polypeptide of interest, e.g. by use of appropriate controls. In general, antibodies used in compositions and methods described herein which bind to a specific protein marker protein with a binding affinity of 10⁷ moles/L or more, preferably 10⁸ moles/L or more are said to bind specifically to the specific protein marker protein.

In embodiments, the affinity of specific binding of an antibody or other binding agent to a protein marker is about 2 times greater than background binding, about 5 times greater than background binding, about 10 times greater than background binding, about 20 times greater than background binding, about 50 times greater than background binding, about 100 times greater than background binding, or about 1000 times greater than background binding, or more.

In embodiments, the affinity of specific binding of an antibody or other binding agent to a protein marker is between about 2 to about 1000 times greater than background binding, between about 2 to 500 times greater than background binding, between about 2 to about 100 times greater than background binding, between about 2 to about 50 times greater than background binding, between about 2 to about 20 times greater than background binding, between about 2 to about 10 times greater than background binding, or any intervening range of affinity.

In embodiments, the concentration of a protein marker is determined using an assay or format including, but not limited to, e.g., immunoassays, ELISA sandwich assays, lateral flow assays, flow cytometry, mass spectrometric detection, calorimetric assays, binding to a protein array (e.g., antibody array), single molecule detection methods, nanotechnology-based detection methods, or fluorescent activated cell sorting (FACS). In some embodiments, an approach involves the use of labeled affinity reagents (e.g., antibodies, small molecules, etc.) that recognize epitopes of one or more protein marker proteins in an immunoassay, an ELISA, antibody-labelled fluorescent bead array, antibody array, or FACS screen. As noted, any of a number of illustrative methods for producing, evaluating and/or using antibodies for detecting and quantifying the protein markers herein are well known and available in the art. It will also be understood that the protein detection and quantification in accordance with the methods described herein can be carried out in single assay format, multiplex format, or other known formats.

In embodiments, the concentration of a given protein is normalized to a quantification standard. In embodiments, the quantification standard is a synthetic. A number of normalization methods are known in the art. In embodiments, the normalized protein concentrations are mathematically transformed. The mathematical transformation can be log-transformation.

A number of suitable high-throughput multiplex formats exist for evaluating the disclosed protein markers. Typically, the term “high-throughput” refers to a format that performs a large number of assays per day, such as at least 100 assays, 1000 assays, up to as many as 10,000 assays or more per day. When enumerating assays, either the number of samples or the number of markers assayed can be considered.

In some embodiments, the samples are analyzed on an assay system or analytical device. For example, the assay system or analytical device may be a multiplex analyzer that simultaneously measures multiple analytes, e.g., proteins, in a single microplate well. The assay format may be receptor-ligand assays, immunoassays, and enzymatic assays. An example of such an analyzer is the Luminex® 100/200 system which is a combination of three xMAP® Technologies. The first is xMAP microspheres, a family of fluorescently dyed micron-sized polystyrene microspheres that act as both the identifier and the solid surface to build the assay. The second is a flow cytometry-based instrument, the Luminex® 100/200 analyzer, which integrates key xMAP® detection components, such as lasers, optics, fluidics, and high-speed digital signal processors. The third component is the xPONENT® software, which is designed for protocol-based data acquisition with robust data regression analysis.

By determining protein marker levels and optionally clinical variable status for a subject, a dataset may be generated and used (as further described herein) to classify the biological sample to one or more of risk stratification, prognosis, diagnosis, and monitoring of the cardiovascular status of the subject, and further assigning a likelihood of a positive, intermediate, or negative diagnosis, outcome, or one or more future changes in cardiovascular status to the subject to thereby establish a diagnosis and/or prognosis of cardiovascular disease and/or outcome, as described herein. The dataset may be obtained via automation or manual methods.

Statistical Analysis

By analyzing combinations of protein markers and optional clinical variables as described herein, the methods described herein are capable of discriminating between different endpoints. The endpoints may include, for example, peripheral artery disease (PAD), limb amputation, and/or aortic stenosis (AS). The identity of the markers and their corresponding features (e.g., concentration, quantitative levels) are used in developing and implementing an analytical process, or plurality of analytical processes, that discriminate between clinically relevant classes of patients.

Methods described herein may utilize machine learning. Machine learning is a field of statistics and computer science where algorithms generate models from data for the sake of prediction, regression, or classification. Machine learning algorithms generally require a set of “features”, which are the variables that are used to predict an “outcome” or “class”. In embodiments herein, the features are the normalized, log-transformed protein concentrations and the clinical factors, and the class or outcome is the medical outcome that we are trying to predict. The accuracy of learning models can be evaluated with many different metrics, depending on the type of class that the model is trying to predict (e.g., different metrics will be used for a binary outcome (e.g., “positive” vs. “negative”) than for a tertiary or continuous numeric outcome (the amount of obstruction present in a given artery). Machine learning gives computers the ability to learn without being explicitly programmed. Machine learning explores the study and construction of algorithms that can learn from and make predictions on data—such algorithms overcome following strictly static program instructions by making data-driven predictions or decisions through building a model from sample inputs. Machine learning is employed in a range of computing tasks where designing and programming explicit algorithms with good performance is difficult or infeasible. As a scientific endeavor, machine learning grew out of the quest for artificial intelligence (AI) and is considered a subset of artificial intelligence. Already in the early days of AI, some researchers were interested in having machines learn from data. They attempted to approach the problem with various symbolic methods, as well as what were then termed “neural networks”. Probabilistic reasoning was also employed, especially in automated medical diagnosis.

A protein marker and clinical variable dataset may be used in an analytic process for correlating the assay result(s) generated by the assay system and optionally the clinical variable status to the cardiovascular status of the subject, wherein said correlation step comprises correlating the assay result(s) to one or more of risk stratification, prognosis, diagnosis, classifying and monitoring of the cardiovascular status of the subject, monitoring of cardiovascular effects of pharmacologic agents, identifying high risk patients for clinical trial enrollment, also referred to as clinical trial enrichment, or use as a companion diagnostic or complementary diagnostic for pharmacologic agents, medical devices or other treatments in patients known of, or suspected of, peripheral artery disease, peripheral limb amputation risk, or aortic stenosis, wherein said correlating step comprises assigning a likelihood of a positive, intermediate, or negative diagnosis, or one or more future changes in cardiovascular status to the subject based on the assay result(s).

A protein marker and clinical variable dataset may be used in an analytic process for generating a diagnostic and/or prognostic result or score. For example, an illustrative analytic process can comprise a linear model with one term for each component (protein level or clinical factor). The result of the model is a number that generates a diagnosis and/or prognosis. The model allows for the establishment of an algorithm for a particular protein marker and/or clinical variable dataset which is then used to generate a score. The result may also provide a multi-level or continuous score with a higher number representing a higher likelihood of disease or risk of event, a lower number representing a lower likelihood of disease or risk of event, and an intermediate number representing an intermediate likelihood of disease or risk of event.

The examples below illustrate how data analysis algorithms can be used to construct a number of such analytical processes. Each of the data analysis algorithms described in the examples uses features (e.g., normalized and transformed quantitative protein levels and/or clinical factors) of a subset of the markers identified herein across a training population. Specific data analysis algorithms for building an analytical process or plurality of analytical processes, that discriminate between subjects disclosed herein will be described in the subsections below. Once an analytical process has been built using these example data analysis algorithms or other techniques known in the art, the analytical process can be used to classify a test subject into one of the two or more phenotypic classes and/or predict survival/mortality or a severe medical event within a specified period of time after the blood test is obtained. This is accomplished by applying one or more analytical processes to one or more marker profile(s) obtained from the test subject. Such analytical processes, therefore, have enormous value as diagnostic or prognostic indicators.

In embodiments, the methods provide for normalization and transformation of the concentrations of a panel of protein markers, as described above, and subsequent use of an algorithm to convert the normalized, transformed concentration data into a score that may be used to determine whether a patient is diagnosed with obstructive peripheral artery disease or aortic valve stenosis or has a prognosis of risk for developing an adverse cardiovascular event, including, but not limited to, peripheral limb amputation and peripheral revascularization.

The data are processed prior to the analytical process. The data in each dataset are collected by measuring the values for each marker, usually in duplicate or triplicate or in multiple replicates. The data may be manipulated; for example, raw data may be transformed using standard curves, and the average of replicate measurements used to calculate the average and standard deviation for each patient. These values may be transformed before being used in the models, e.g., log-transformed, normalized to a standard scale, Winsorized, etc. The data is transformed via computer software. This data can then be input into the analytical process with defined parameters.

The direct concentrations of the proteins (after log-transformation and normalization), the presence/absence of clinical factors represented in binary form (e.g., sex), and/or clinical factors in quantitative form (e.g., BMI, age) provide values that are entered into the algorithmically-weighted diagnostic and/or prognostic model provided by the software, and the result is evaluated against one or more cutoffs to determine the diagnosis or prognosis.

The following are examples of the types of statistical analysis methods that are available to one of skill in the art to aid in the practice of the disclosed methods, panels, assays, and kits. The statistical analysis may be applied for one or both of the two following tasks: (1) these and other statistical methods may be used to identify preferred subsets of markers and other indices that will form a preferred dataset; (2) these and other statistical methods may be used to generate the analytical process that will be used with the dataset to generate the result. Several statistical methods presented herein or otherwise available in the art will perform both of these tasks and yield a model that is suitable for use as an analytical process for the practice of the methods disclosed herein.

Prior to analysis, the data is partitioned into a training set and a validation set. The training set is used to train, evaluate and build the final diagnostic or prognostic model. The validation set is not used at all during the training process, and is only used to validate final diagnostic or prognostic models.

The creation of training and validation sets can be done through random selection, or through chronological selection (i.e., where the training set is the first sequential set of patients, and the validation set is the second/final sequential set of patients). After these sets are determined, the balance of various outcomes (e.g., presence of 50% or greater obstruction, risk of peripheral limb amputation, etc.) is considered to confirm that the outcomes of interest are properly represented in each data set.

In cases where sample sizes are small, the entire population of patients is used to train, evaluate, and develop a diagnostic or prognostic panel. All processes below, except when explicitly mentioned, involve the use of the entire population.

The features (e.g., proteins and/or clinical factors) of the diagnostic and/or prognostic models are selected for each outcome using a combination of analytic processes, including least angle regression (LARS; a procedure based on stepwise forward selection), shrinkage in statistical learning methods such as least absolute shrinkage and selection operator (LASSO), significance testing, and expert opinion.

The statistical learning method used to generate a result (classification, diagnosis, and/or disease/outcome risk within a specified time, etc.) may be any type of process capable of providing a result useful for classifying a sample (e.g., a linear model, a probabilistic model, a decision tree algorithm, or a comparison of the obtained dataset with a reference dataset).

The diagnostic or prognostic signal in the features is evaluated with these statistical learning methods using a cross-validation procedure. For each cross-validation fold, the data (either the training set or all patients, depending on the sample size) is further split into training and validation sets (hereby called CV-training and CV-validation data sets).

For each fold of cross validation, the diagnostic or prognostic model is built using the CV-training data, and evaluated with the CV-validation data.

Models during the cross-validation process are evaluated with standard metrics of classification accuracy, e.g., the area under the ROC curve (AUC), sensitivity (Sn), specificity (Sp), positive predictive values (PPV), and negative predictive values (NPV).

Once a set of features (e.g., quantitative protein levels and optionally clinical factors) are selected to compose a final diagnostic or prognostic panel, a final predictive model is built using all of the training data.

Applying the patient data (e.g., transformed and normalized quantitative protein levels and/or clinical factors) into the final predictive model yields a classification result. These results can be compared against a threshold for classifying a sample within a certain class (e.g., positive, intermediate, or negative diagnosis and/or prognosis, or a severity/likelihood score).

For small populations, a final model is created with the entire population, and then this model is evaluated again with the population to determine the in-sample diagnostic or prognostic results.

For populations of sufficient size to warrant separation into training and validation sets, final models are evaluated with the validation data set. To respect the authority of the validation data set, it is not used in an iterative way, to feed information back into the training process. It is only used as the final step of the analytic pipeline.

Models are evaluated with the entire population (for smaller populations) or with the validation data set (for populations of sufficient size to warrant separation into training and validation set), using metrics of diagnostic accuracy, including the AUC, sensitivity, specificity, positive predictive value and/or negative predictive value. Other metrics of accuracy, such as hazard ratio, relative risk, and net reclassification index are considered separately for models of interest.

This final model or a model optimized for a particular protein marker platform, when used in a clinical setting, may be implemented as a software system, running directly on the assay hardware platform or on an independent system. The model may receive protein level or concentration data directly from the assay platform or other means of data transfer, and patient clinical data may be received via electronic, manual, or other query of patient medical records or through interactive input with the operator. This patient data may be processed and run through the final model, which will provide a result to clinicians and medical staff for purposes of decision support.

In embodiments, the protein markers and/or clinical variables include those listed in Table 1, particularly those that are associated with a p-value of less than 0.1, less than 0.05, less than 0.01 or less than 0.001.

In some embodiments, at least 2, at least 3 or at least 4 protein markers are used in the methods provided herein. In other embodiments, the number of protein markers employed can vary, and may include at least 5, 6, 7, 8, 9, 10, or more. In still other embodiments, the number of protein markers can include at least 15, 20, 25 or 50, or more.

In embodiments, the methods provided herein include measuring the concentrations of at least two protein markers selected from Table 1. In embodiments, the methods provided herein include measuring the concentrations of at two protein markers selected from Table 1. In embodiments, the methods provided herein include measuring the concentrations of three protein markers selected from Table 1. In embodiments, the methods provided herein include measuring the concentrations of four protein markers selected from Table 1. In embodiments, the methods provided herein include measuring the concentrations of five protein markers selected from Table 1. In embodiments, the methods provided herein include measuring the concentrations of six protein markers selected from Table 1. Such determination can be made by standard methods known in the art and described herein. In embodiments, measurement of the concentrations of the protein markers selected from Table 1 determines a subject's protein profile.

In embodiments, the analytical device for measuring the concentrations of protein markers is an immunoassay device. The device may be configured with software controls and analytical programs capable of mathematical computations such as normalizing detected protein marker concentrations against a quantification standard. The quantification standard may be part of the protein detection assay or may be separately contained. The software controls and analytical programs may be further capable of receiving clinical variables entered as a mathematical factor and log-transforming the normalized protein concentrations into a value that is then converted into a score based on pre-entered algorithms and models to accept the protein marker concentrations and the optional clinical variable(s). The mathematical log-transformations and use of an algorithm to generate a diagnostic and/or prognostic score can be accomplished within the analytical device, a computer, in a cloud computing setting or the like.

In embodiments, the status of at least one clinical variable or measurement selected from Table 2 is determined. In embodiments, the methods provided herein include determining the status of one clinical variable selected from Table 2. In embodiments, the methods provided herein include determining the status of two clinical variables selected from Table 2. In embodiments, the methods provided herein include determining the status of three clinical variable selected from Table 2. In embodiments, the methods provided herein include determining the status of four clinical variable selected from Table 2. Such determination can be made by standard methods known in the art such as medical history review or from the analytical device retrieving the clinical variable(s) from other means, including but not limited to electronic health records (EHR) or other information systems, or clinical lab tests.

In embodiments, assigning a score to the subject based on the protein marker profile and optionally the clinical value status can be accomplished using a device configured with software controls and analytical programs capable of mathematical computations as described above. The score may be classified as a positive, intermediate, or negative diagnostic result. The score may be classified as a positive, intermediate, or negative prognostic result.

Scoring and Treatments

In some embodiments, the diagnostic or prognostic calculations will result in a numeric or categorical score that relates the patient's level of likelihood of PAD, e.g., including but not limited to positive predictive value (PPV), negative predictive value (NPV), sensitivity (Sn), or specificity (Sp), and/or the risk of a cardiovascular event occurring within the specified period. The number of levels used by the diagnostic model may be as few as two (“positive” vs. “negative”) or as many as deemed clinically relevant, e.g., a diagnostic model for PAD may result a five-level score, where a higher score indicates a higher likelihood of disease. Specifically, a score of 1 indicates a strong degree of confidence in a low likelihood of PAD or a negative result (determined by the test's NPV or Sn), a score of 5 indicates a strong degree of confidence in a high likelihood of PAD or a positive result (determined by the test's PPV or Sp), and a score of 3 indicates an intermediate or moderate likelihood for PAD.

In embodiments, the methods provided herein further include treating the subject based on a positive, intermediate or negative diagnostic score for peripheral artery disease. Treating the subject includes providing a therapeutic regimen. The therapeutic regimen may include administration of therapeutic drugs, further diagnostic testing, lifestyle modification, surgical intervention and the like. In embodiments, a positive diagnostic score in the subject facilitates a determination by a medical practitioner of the need for one or more interventions selected from ultrasound, administration of pharmacological agents, peripheral angiography, peripheral revascularization (peripheral angioplasty, stent or bypass), and avoidance of any drug with a known amputation risk. In embodiments, a negative diagnostic score in the subject facilitates a determination by a medical practitioner of the need for one or more interventions selected from ongoing monitoring and management of peripheral and coronary risk factors, and lifestyle modifications. In embodiments, an intermediate diagnostic score in the subject facilitates a determination by a medical practitioner of the need for one or more interventions selected from further testing, arterial brachial index (ABI) testing, and more frequent monitoring for risk factors.

In embodiments, the methods provided herein further include treating the subject based on a positive, intermediate or negative prognostic score for risk of peripheral limb amputation. Treating the subject includes providing a therapeutic regimen. The therapeutic regimen may include administration of therapeutic drugs, avoidance of any pharmacologic agents with a known amputation risk, selection of pharmacologic agents that may help in other disease states which may or may not have a risk of amputation, further diagnostic testing, complementary diagnostic or companion diagnostic testing, lifestyle modification, peripheral angiography, surgical intervention including peripheral revascularization (balloon, stent or bypass), ultrasound, more frequent monitoring for risk factors such as diabetes and high cholesterol, and the like. In embodiments, a positive prognostic score in the subject facilitates a determination by a medical practitioner of the need for one or more interventions selected from ultrasound, administration of pharmacological agents, avoidance of any pharmacologic agents with a known amputation risk, peripheral angiography, peripheral revascularization (balloon, stent or bypass), complementary diagnostic or companion diagnostic testing, and lifestyle modifications. In embodiments, a negative prognostic score in the subject facilitates a determination by a medical practitioner of the need for one or more interventions selected from one or more of ongoing monitoring and management of peripheral and coronary risk factors including hypertension, diabetes, and smoking, selection of pharmacologic agents that may help in other disease states which may or may not have a risk of amputation, and lifestyle modifications. In embodiments, an intermediate prognostic score in the subject facilitates a determination by a medical practitioner of the need for one or more interventions selected from one or more of arterial brachial index (ABI) testing, selection of pharmacologic agents which may or may not have a risk of amputation, and more frequent monitoring for risk factors such as diabetes and high cholesterol.

In some embodiments, the diagnostic or prognostic model will result in a numeric or categorical score that relates the patient's level of likelihood of aortic stenosis (AS), e.g. including but not limited to positive predictive value (PPV), negative predictive value (NPV), sensitivity (Sn), or specificity (Sp) or the risk of a cardiovascular event occurring within the specified time. The number of levels used by the diagnostic model may be as few as two (“positive” vs. “negative”) or as many as deemed clinically relevant, e.g., a diagnostic model for AS may result a five-level score, where a higher score indicates a higher likelihood of disease. Specifically, a score of 1 indicates a strong degree of confidence in a low likelihood of AS or a negative result (determined by the test's NPV or Sn), a score of 5 indicates a strong degree of confidence in a high likelihood of AS or a positive result (determined by the test's PPV or Sp), and a score of 3 indicates an intermediate or moderate likelihood for AS.

In embodiments, the methods provided herein further include treating the subject based on a positive, intermediate or negative diagnostic score for aortic valve stenosis. Treating the subject includes providing a therapeutic regimen. The therapeutic regimen may include administration of further diagnostic testing including but not limited to echocardiogram, electrocardiogram (ECG), chest x-ray, cardiac computed tomography (CT), cardiac magnetic resonance imaging (MRI), exercise stress testing, surgical intervention including aortic valve repair or replacement, ongoing monitoring, education symptomatology, lifestyle modification and treatment of diabetes, high cholesterol and high blood pressure, and more frequent monitoring and/or physician visits.

In embodiments, a positive diagnostic score in the subject facilitates a determination by a medical practitioner of the need for one or more interventions selected from echocardiogram, electrocardiogram (ECG), chest x-ray, cardiac computed tomography (CT), cardiac magnetic resonance imaging (MRI), surgical aortic valve repair or replacement. In embodiments, a negative diagnostic score in the subject facilitates a determination by a medical practitioner of the need for one or more interventions selected from ongoing monitoring, education symptomatology, and lifestyle modifications, including, but not limited to, healthy diet, maintaining healthy weight, regular physical activity, cessation of smoking, managing stress. In embodiments, an intermediate diagnostic score in the subject facilitates a determination by a medical practitioner of the need for one or more interventions selected from exercise stress testing, further testing, more frequent monitoring for risk factors and/or physician visits.

Panels, Assays, and Kits

The present disclosure further provides panels, assays, and kits comprising target-binding agents that bind at least 2, at least 3, at least 4 or greater than 4 protein markers and optionally clinical variable(s), in order to aid or facilitate a diagnostic or prognostic finding according to the present disclosure. For example, in some embodiments, a diagnostic or prognostic panel or kit comprises one or a plurality of protein markers set out in Table 1 and optionally one or a plurality of applicable clinical variables set out in Table 2.

It will be understood that, in many embodiments, the panels, assays, and kits described herein comprise antibodies, binding fragments thereof and/or other types of binding agents which are specific for the protein markers of Table 1, and which are useful for determining the concentrations of the corresponding protein marker in a biological sample according to the methods describe herein. Accordingly, in each description herein of a panel, assay, or kit comprising one or a plurality of protein markers, it will be understood that the very same panel, assay, or kit can advantageously comprise, in addition or instead, one or a plurality of antibodies, binding fragments thereof or other types of target binding agents such as aptamers, which are specific for a protein marker as set forth in Table 1. Of course, the panels, assays, and kits can further comprise, include or recommend a determination of one or a plurality of applicable clinical variables as set out in Table 2.

In certain specific embodiments, the protein markers and/or clinical variables used in conjunction with a panel, assay, or kit include those listed in Table 1 and Table 2 respectively, particularly those which are associated with a p-value of less than 0.1, less than 0.05, less than 0.01 or less than 0.001.

In some embodiments, panels, assays, and kits may comprise at least 2, at least 3 or at least 4 target-binding agents specific for protein markers as described herein. In embodiments, panels, assays, and kits may comprise target-binding agents for two protein markers. In embodiments, panels, assays, and kits may comprise target-binding agents for three protein markers. In embodiments, panels, assays, and kits may comprise target-binding agents for four protein markers. In embodiments, panels, assays, and kits may comprise target-binding agents for five protein markers. In other embodiments, the number of protein markers employed can include at least 5, 6, 7, 8, 9 or 10 or more. In still other embodiments, the number of protein markers employed can include at least 15, 20, 25 or 50, or more.

As described herein, panels, assays, and kits of the present disclosure can be used for identifying the presence of cardiovascular disease in a subject, particularly the presence of obstructive peripheral artery disease and/or for predicting cardiovascular events. In some embodiments, a diagnostic panel, assay, or kit identifies in a subject the presence of 50% or greater obstruction in a peripheral artery.

In other embodiments, a prognostic panel, assay, or kit is used to predict the risk of a cardiovascular disease or event within one year, about 1 year, about 2 years, about 3 years, about 4 years, about 5 years, or more from the date on which the sample is drawn. Time endpoints are defined as from sample draw and include less than one year, one year, and greater than one year. Less than or within one year may be any time from time of sample draw up to and including 365 days. For example, the panel results may predict the risk of a cardiovascular disease or event from time of sample draw to 30 days, to 60 days, to 90 days, to 120 days, to 150 days, to 180 days, to 210 days, to 240 days, to 270 days, to 300 days, to 330 days, to 360 days, to 365 days. In yet other embodiments, time endpoints are defined as 3 days post sample draw to 30 days, 3 days to 60 days, 3 days to 90 days, 3 days to 120 days, 3 days to 150 days, 3 days to 180 days, 3 days to 210 days, 3 days to 240 days, 3 days to 270 days, 3 days to 300 days, 3 days to 330 days, 3 days to 360 days, to 3 days 365 days. Suitable time frames include any value or subrange within the recited range, including endpoints.

In specific embodiments, panels, assays, and kits for the diagnosis of peripheral artery disease (PAD) and/or prognosis of peripheral revascularization and/or monitoring PAD progression or therapeutic effect and/or prognosis of peripheral limb amputation comprise at least 2, at least 3, at least 4 or greater than four protein markers, or antibodies, binding fragments thereof or other types of binding agents, which are specific for the protein markers, where the protein markers are selected from, angiopoietin 1, apolipoprotein C-I, angiotensin converting enzyme, carcinoembryonic antigen related cell adhesion molecule 1, eotaxin 1, ENRAGE, fetuin A, follicle stimulating hormone, intercellular adhesion molecule 1, interferon gamma induced protein 10, interleukin 1 receptor antagonist, interleukin 8, interleukin 23, kidney injury molecule 1, matrix metalloproteinase 7, matrix metalloproteinase 9 Total, midkine, monokine induced by gamma interferon, myeloid progenitor inhibitory factor 1, osteopontin, pulmonary surfactant associated protein D, resistin, serotransferrin, Tamm Horsfall urinary glycoprotein, T Cell specific protein RANTES, thyroxine binding globulin, and transthyretin. In some embodiments, at least one clinical variable described herein is used in conjunction with the protein marker levels determined. In other embodiments, the clinical variable is selected from age, history of hypertenstion, history of peripheral percutaneous angioplasty (with or without stent), body mass index (BMI), history of dyslipidemia, and/or history of peripheral revascularization (peripheral angioplasty, stent or bypass.

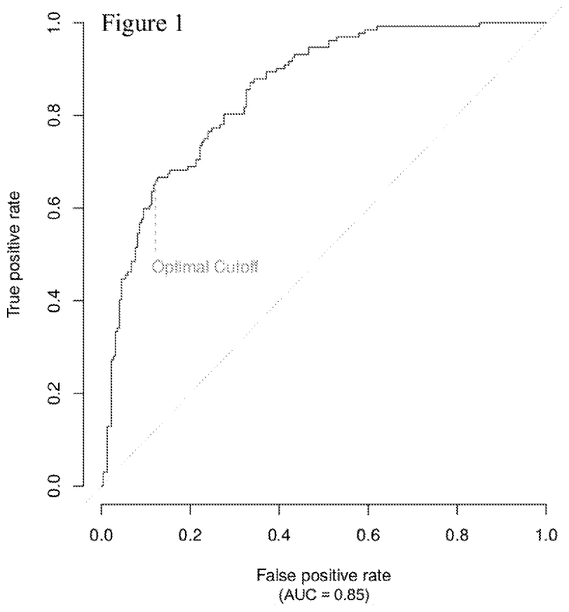

In specific embodiments, a panel, assay, or kit for the diagnosis of 50% or greater obstruction in a peripheral artery and/or monitoring PAD progression or therapeutic effect comprises protein markers for angiopoietin 1, eotaxin 1, follicle stimulating hormone, interleukin 23, kidney injury molecule 1, and midkine and clinical variables of history of hypertension. This combination of protein markers and clinical variables is represented by panel PAD158 in Table 3, Example 1, FIGS. 1, 5, and 7.

In specific embodiments, a panel, assay, or kit for the diagnosis of 50% or greater obstruction in a peripheral artery and/or monitoring PAD progression or therapeutic effect comprises protein markers kidney injury molecule 1, interleukin 1 receptor antagonist, pulmonary surfactant associated protein D, and clinical variable of history of percutaneous peripheral angioplasty (with or without stent). This combination of protein markers is represented by panel PAD027VA in Table 3.

In specific embodiments, a panel, assay, or kit for the diagnosis of 50% or greater obstruction in a peripheral artery and/or monitoring PAD progression or therapeutic effect comprises protein markers for serotransferrin, T Cell Specific Protein RANTES, thyroxine binding globulin, and transthyretin. This combination of protein markers is represented by panel PAD104 in Table 3.

In another embodiment, a panel, assay, or kit for the diagnosis of 50% or greater obstruction in a peripheral artery and/or monitoring PAD progression or therapeutic effect comprises protein markers for serotransferrin, T Cell Specific Protein RANTES, thyroxine binding globulin, and transthyretin and clinical variable of history of hypertension. This combination of protein markers and clinical variables is represented by panel PAD103 in Table 3.

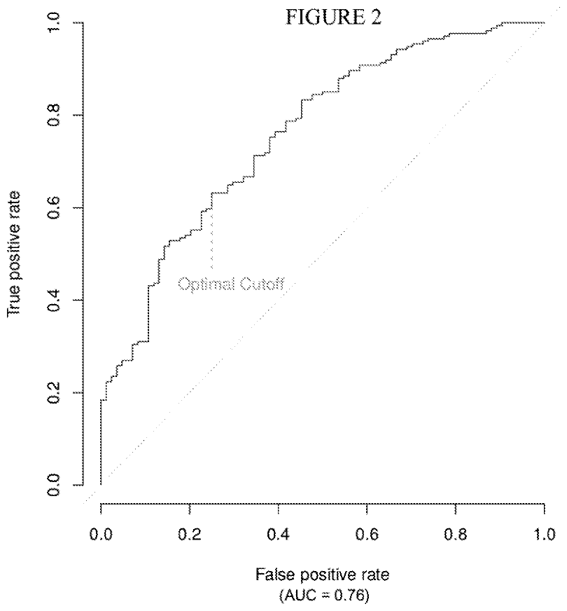

In another embodiment, a panel, assay, or kit for the diagnosis of 50% or greater obstruction in a peripheral artery and/or monitoring PAD progression or therapeutic effect comprises protein markers fetuin A, interleukin 8, kidney injury molecule 1, osteopontin, T Cell Specific Protein RANTES, and Tamm Horsfall urinary glycoprotein and clinical variables of body mass index and history of hypertension. This combination of protein markers is represented by panel PAD076 in Table 3, Example 2, and FIGS. 2, 6, and 8.

In another embodiment, a panel, assay, or kit for the diagnosis of 50% or greater obstruction in a peripheral artery and/or monitoring PAD progression or therapeutic effect comprises protein markers for angiopoietin 1, eotaxin 1, follicle stimulating hormone, interleukin 23, kidney injury molecule 1, and midkine, and clinical variables of history of dyslipidemia and history of hypertension. This combination of protein markers and clinical variables is represented by panel PAD157 in Table 3.

In another embodiment, a panel, assay, or kit for the diagnosis of 50% or greater obstruction in a peripheral artery and/or monitoring PAD progression or therapeutic effect comprises protein markers for angiopoietin 1, apolipoprotein Cl, eotaxin 1, follicle stimulating hormone, interleukin 23, kidney injury molecule 1, matrix metalloproteinase 7, midkine, and Tamm Horsfall urinary glycoprotein and clinical variables of history of dyslipidemia and history of hypertension. This combination of protein markers and clinical variables is represented by panel PAD154 in Table 3.

In another embodiment, a panel, assay, or kit for the diagnosis of 50% or greater obstruction in a peripheral artery and/or monitoring PAD progression or therapeutic effect comprises protein markers for angiopoietin 1, eotaxin 1, fetuin A, interleukin 23, and kidney injury molecule 1, and clinical variable of history of hypertension. This combination of protein markers and clinical variables is represented by panel PAD145 in Table 3.

Embodiments of the present disclosure comprise a panels, assays, and kits for the diagnosis of 50% or greater obstruction in a peripheral artery and/or prognosis of PAD amputation or need for peripheral intervention (balloon, stent or bypass) and/or monitoring therapeutic effects comprising at least one protein marker and one or more clinical variables.

In a specific embodiment, a panel, assay, or kit for the diagnosis of 50% or greater obstruction in a peripheral artery and/or need for peripheral intervention and/or monitoring PAD progression or therapeutic and/or prognosis of peripheral limb amputation risk comprises a protein marker for angiotensin converting enzyme, carcinoembryonic antigen related cell adhesion molecule 1, interferon gamma induced protein 10, osteopontin, pulmonary surfactant associated protein D, T Cell Specific Protein RANTES, and Tamm Horsfall urinary glycoprotein and clinical variables of history of peripheral revascularization intervention (peripheral angioplasty, stent or bypass) and history of hypertension. This combination of protein markers and clinical variables is represented by panel PAD001 in Table 3.

In a specific embodiment, a panel, assay, or kit for the diagnosis 50% or greater obstruction in a peripheral artery and/or need for peripheral intervention and/or monitoring PAD progression or therapeutic effect and/or prognosis of peripheral limb amputation risk comprises a protein marker for angiotensin converting enzyme, kidney injury molecule 1, osteopontin, and pulmonary surfactant associated protein D and clinical variable of history of peripheral revascularization intervention (peripheral angioplasty, stent or bypass). This combination of protein markers and clinical variables is represented by panel PAD010 in Table 3.

In a specific embodiment, a panel, assay, or kit for the prognosis of peripheral revascularization (balloon, stent or bypass) or peripheral limb amputation risk comprises a protein marker for angiotensin converting enzyme, ENRAGE, intercellular adhesion molecule 1, monokine induced by gamma interferon, myeloid progenitor inhibitory factor 1, pulmonary surfactant associated protein D, and resistin and clinical variable of age. This combination of protein markers and clinical variables is represented by panel PAD026 in Table 3.

In a specific embodiment, a panel, assay, or kit for the prognosis of peripheral revascularization (balloon, stent or bypass) or peripheral limb amputation risk comprises a protein marker for matrix metalloproteinase 7, matrix metalloproteinase 9 Total, myeloid progenitor inhibitory factor 1, pulmonary surfactant associated protein D, and resistin and clinical variable of history of peripheral revascularization intervention (peripheral angioplasty, stent or bypass). This combination of protein markers and clinical variables is represented by panel PAD018 in Table 3.

Embodiments of the present disclosure also provide panels, assays, and kits for the prognosis of peripheral limb amputation, where the panels comprise one or more protein markers or antibodies, binding fragments thereof or other types of binding agents, which are specific for the protein markers disclosed herein. Such panels, assays, and kits can be used, for example, for determining a prognosis of the risk of a peripheral limb amputation within a specified time in the subject, such as within one year, or within three years, or within five years. In some embodiments, the time endpoint is defined as starting from sample draw. In specific embodiments, panels, assays, and kits for the prognosis of peripheral limb amputation comprise at least 2, at least 3, at least 4 or greater than four protein markers, or antibodies, binding fragments thereof or other types of binding agents, which are specific for the protein markers, where the protein markers are selected from Factor VII, ferritin, growth hormone, immunoglobulin M, kidney injury molecule 1, and vitamin D binding protein. In embodiments, the methods include determining the clinical variable of history of diabetes mellitus type 2 and/or smoker.

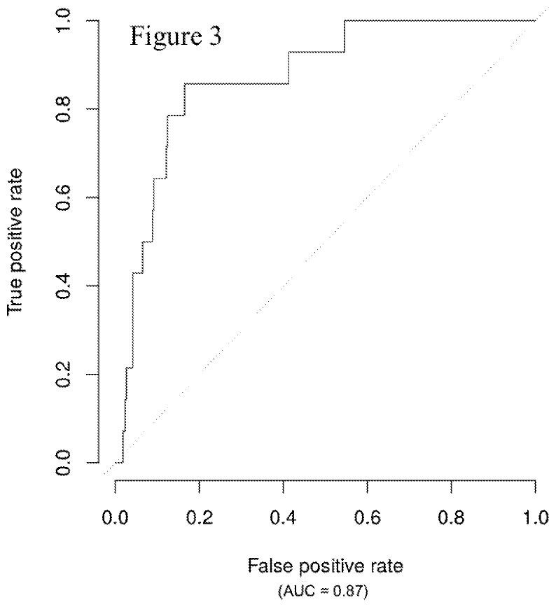

In certain specific embodiments, a panel, assay, or kit for the prognosis of a peripheral limb amputation comprises the protein markers kidney injury molecule-1 and vitamin D binding protein, and the clinical variable of history of diabetes mellitus type 2. This combination of protein markers and clinical variable is represented by panel AMPU018 in Table 3, Example 3, and FIG. 3.

In certain specific embodiments, a panel, assay, or kit for the prognosis of a peripheral limb amputation comprises the protein markers factor VII and vitamin D binding protein and the clinical variables of history of diabetes mellitus type 2 and smoker. This combination of protein markers and clinical variable is represented by panel AMPU010 in Table 3.

In certain specific embodiments, a panel, assay, or kit for the prognosis of a peripheral limb amputation comprises the protein markers factor VII, growth hormone and vitamin D binding protein and the clinical variables of history of diabetes mellitus type 2 and smoker. This combination of protein markers and clinical variable is represented by panel AMPU008 in Table 3.

In certain specific embodiments, a panel, assay, or kit for the prognosis of a peripheral limb amputation comprises the protein markers factor VII, ferritin, growth hormone, immunoglobulin M, and vitamin D binding protein and the clinical variable of history of diabetes mellitus type 2. This combination of protein markers and clinical variable is represented by panel AMPU013 in Table 3.

Embodiments of the present disclosure also provide panels, assays, and kits for the diagnosis of aortic valve stenosis where the panels comprise one or more protein markers or antibodies, binding fragments thereof or other types of binding agents, which are specific for the protein markers disclosed herein. In specific embodiments, panels, assays, and kits for the diagnosis of aortic valve stenosis comprise at least 2, at least 3, at least 4 or greater than four protein markers, or antibodies, binding fragments thereof or other types of binding agents, which are specific for the protein markers, where the protein markers are selected from adiponectin, Apolipoprotein(a), fetuin A, interleukin 18, N terminal prohormone of brain natriuretic peptide, osteopontin, resistin, and von Willebrand factor. In embodiments, the methods include determining the clinical variable of age and/or history of coronary revascularization intervention (coronary angioplasty, stent or bypass).

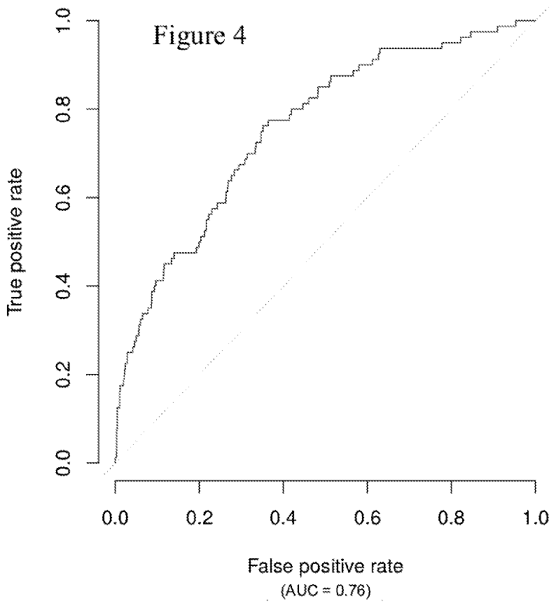

In certain specific embodiments, a panel, assay, or kit for the diagnosis of aortic valve stenosis comprises protein markers for fetuin A, N terminal prohormone of brain natriuretic peptide, and von Willebrand factor and the clinical variable of age. This combination of protein markers is represented by panel ASR025 in Table 3, Example 4 and FIG. 4.

In one specific embodiment, a panel, assay, or kit for the diagnosis of aortic valve stenosis comprises protein markers for adiponectin, apolipoprotein(a), interleukin 18, N terminal prohormone of brain natriuretic peptide, resistin, and von Willebrand Factor and the clinical variables of age and history of coronary revascularization intervention (coronary angioplasty, stent or bypass). This combination of protein markers is represented by panel ASR001 in Table 3.

In one specific embodiment, a panel, assay, or kit for the diagnosis of aortic valve stenosis comprises protein markers for adiponectin, apolipoprotein(a), interleukin 18, N terminal prohormone of brain natriuretic peptide, resistin, and von Willebrand factor and the clinical variable of age. This combination of protein markers is represented by panel ASR002 in Table 3.

In one specific embodiment, a panel, assay, or kit for the diagnosis of aortic valve stenosis comprises protein markers for apolipoprotein(a), N terminal prohormone of brain natriuretic peptide, resistin, and von Willebrand factor and the clinical variable of age. This combination of protein markers is represented by panel ASR003 in Table 3.

In one specific embodiment, a panel, assay, or kit for the diagnosis of aortic valve stenosis comprises protein markers for apolipoprotein(a), N terminal prohormone of brain natriuretic peptide, and von Willebrand factor and the clinical variable of age. This combination of protein markers is represented by panel ASR016 in Table 3.

In one specific embodiment, a panel, assay, or kit for the diagnosis of aortic valve stenosis comprises protein markers for N terminal prohormone of brain natriuretic peptide, resistin, and von Willebrand factor and the clinical variable of age. This combination of protein markers is represented by panel ASR004 in Table 3.

In one specific embodiment, a panel, assay, or kit for the diagnosis of aortic valve stenosis comprises protein markers for adiponectin, N terminal prohormone of brain natriuretic peptide, and von Willebrand factor and the clinical variable of age. This combination of protein markers is represented by panel ASR013 in Table 3.

In one specific embodiment, a panel, assay, or kit for the diagnosis of aortic valve stenosis comprises protein markers for N terminal prohormone of brain natriuretic peptide, osteopontin, and von Willebrand factor and the clinical variable of age. This combination of protein markers is represented by panel ASR006 in Table 3.

In certain embodiments, a panel, assay, or kit comprises at least 2, at least 3, at least 4 or greater than 4 antibodies or binding fragments thereof, or other types of binding agents, where the antibodies, binding fragments or other binding agents are specific for a protein marker of Tablel.

It will be understood that the panels, assays, and kits of the present disclosure may further comprise virtually any other compounds, compositions, components, instructions, or the like, that may be necessary or desired in facilitating a determination of a diagnosis or prognosis according to the present disclosure. These may include instructions for using the panel, assay, or kit, instructions for making a diagnostic or prognostic determination (e.g., by calculating a diagnostic or prognostic score), instructions or other recommendations for a medical practitioner in relation to preferred or desired modes of therapeutic or diagnostic intervention in the subject in light of the diagnostic or prognostic determination, and/or monitoring therapeutic effects and the like.

In some embodiments, the panels, assays, and kits as described herein will facilitate detection of the protein markers discussed herein. Means for measuring such blood, plasma and/or serum concentrations are known in the art, and include, for example, the use of an immunoassay.

In addition to the methods described above, any method known in the art for quantitatively measuring levels of protein in a sample, e.g., non-antibody-based methods can be used in the methods and kits as described herein. For example, mass spectrometry-based (such as, for example, Multiple Reaction Monitoring (MRM) mass spectrometry) or HPLC-based methods can be used. Methods of protein quantification [described in 46-51].

Additionally, technologies such as those used in the field of proteomics and other areas may also be embodied in methods, kits and other aspects as described herein. Such technologies include, for example, the use of micro- and nano-fluidic chips, biosensors and other technologies as described, for example, in U.S. patent application Nos. US2008/0202927; US2014/0256573; US2016/0153980; WO2016/001795; US2008/0185295; US2010/0047901; US2010/0231242; US2011/0154648; US2013/0306491; US2010/0329929; US2013/0261009; [63-70], each of which is incorporated herein by reference in its entirety.

### EXAMPLES

### Example 1: A Clinical and Protein marker Scoring System to Diagnose Peripheral Artery Disease (PAD), Panel PAD158

A convenience sample of 1251 patients undergoing coronary and/or peripheral angiography with or without intervention between 2008 and 2011 were prospectively enrolled. A chronological subset of the final 171 patients were withheld from this analysis, for their potential use in further validation of these models. Additionally, only patients who (a) received a peripheral catheterization and did not receive a coronary catheterization, or (b) received a peripheral catheterization and a coronary catheterization, but had a maximum coronary obstruction of less than 30%, or (c) only received a coronary catheterization, had a maximum coronary obstruction of less than 30%, and had no history of PAD were included in this analysis (n=353), to minimize confounding protein marker signals associated with coronary artery disease. Patients in the category (c) were assumed to be negative for PAD obstruction, using their lack of history of PAD as a surrogate for peripheral obstruction. Patients were referred for these procedures for numerous reasons; this includes angiography following symptoms indicative of PAD and/or coronary artery disease, or pre-operatively prior to heart valve surgery.

After informed consent was obtained, detailed clinical and historical variables and reason for referral for angiography were recorded at the time of the procedure. Results of coronary and/or peripheral angiography were also recorded with highest percent stenosis within each major artery or their branches. For the purposes of this analysis, significant peripheral stenosis was characterized as ≥50% luminal obstruction.

Medical record review from time of enrollment to end of follow up was undertaken. For identification of clinical end points, review of medical records as well as phone follow up with patients and/or managing physicians was performed. The Social Security Death Index and/or postings of death announcements were used to confirm vital status. The following clinical end events were identified, adjudicated, and recorded by study investigators: death, non-fatal MI, HF, stroke, transient ischemic attack, peripheral arterial complication including peripheral limb amputation and/or need for coronary or peripheral revascularization and cardiac arrhythmia. For any recurring events, each discrete event was recorded. Additionally, deaths were adjudicated for presence/absence of a cardiovascular cause.

Fifteen (15) milliliters (mL) of blood was obtained immediately before and immediately after the angiographic procedure through a centrally-placed vascular access sheath. The blood was immediately centrifuged for 15 minutes, serum and plasma aliquoted on ice and frozen at −80° C. until protein marker measurement. Only the blood obtained immediately before the procedure was used for this analysis.

After a single freeze-thaw cycle, 200 microliters (μl) of plasma was analyzed for more than 100 protein markers on a Luminex 100/200 xMAP technology platform. This technology utilizes multiplexed, microsphere-based assays in a single reaction vessel. It combines optical classification schemes, biochemical assays, flow cytometry and advanced digital signal processing hardware and software. Multiplexing is accomplished by assigning each protein-specific assay a microsphere set labeled with a unique fluorescence signature. An assay-specific capture antibody is conjugated covalently to each unique set of microspheres. The assay-specific capture antibody on each microsphere binds the protein of interest. A cocktail of assay-specific, biotinylated detecting antibodies is reacted with the microsphere mixture, followed by a streptavidin-labeled fluorescent “reporter” molecule. Similar to a flow cytometer, as each individual microsphere passes through a series of excitation beams, it is analyzed for size, encoded fluorescence signature and the amount of fluorescence generated is proportionate to the protein level. A minimum of 100 individual microspheres from each unique set are analyzed and the median value of the protein-specific fluorescence is logged. Using internal controls of known quantity, sensitive and quantitative results are achieved with precision enhanced by the analysis of 100 microspheres per data point.

The patients selected for analysis consisted of the chronologically initial 1073 patients who received a coronary and/or peripheral angiogram, and of these, the final 353 patients were selected who also passed the PAD inclusion criteria listed above.

Because of the relatively small number of patients available, all of them were selected to be used for analysis. (i.e., they were not partitioned into a training and a validation set.) Baseline clinical characteristics and protein concentrations between those with and without ≥50% peripheral obstruction in at least one major peripheral artery were compared; dichotomous variables were compared using two-sided Fishers exact test, while continuous variables were compared using two-sided two-sample T test. The protein markers compared were tested with the Wilcoxon Rank Sum test, as their concentrations were not normally distributed. For any marker result that was unmeasurable, we utilized a standard approach of imputing concentrations 50% below the limit of detection.

All work for protein marker selection and the development of a diagnostic model was done on all of the 353 patients that were eligible for the analysis. The level or concentration values for all proteins underwent the following transformation to facilitate the predictive analysis: (a) they were log-transformed to achieve a normal distribution; (b) outliers were clipped at the value of three times the median absolute deviation; and (c) the values were re-scaled to distribution with a zero mean and unit variance. Machine learning statistical techniques, a subset of artificial intelligence, was utilized. Candidate panels of proteins from Table 1 and clinical features from Table 2 were selected via least angle regression (LARS), and models were generated using least absolute shrinkage and selection operator (LASSO) with logistic regression, using Monte Carlo cross-validation with 400 iterations. Candidates were subjected to further assessment of discrimination via iterative model building, assessing change in area under the curve (AUC) with the addition of protein markers to the base model, along with assessment of improvement in calibration from their addition through minimization of the Akaike or Bayesian Information Criteria (AIC, BIC) and goodness of fit in Hosmer-Lemeshow testing.

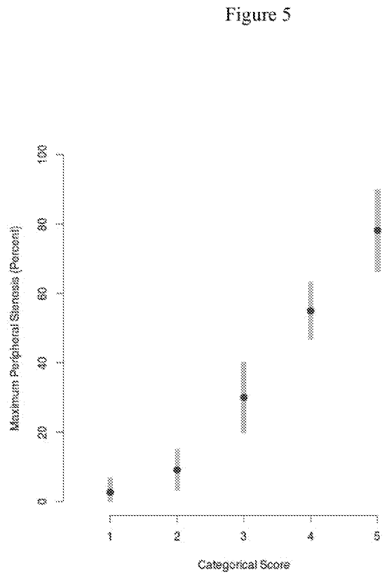

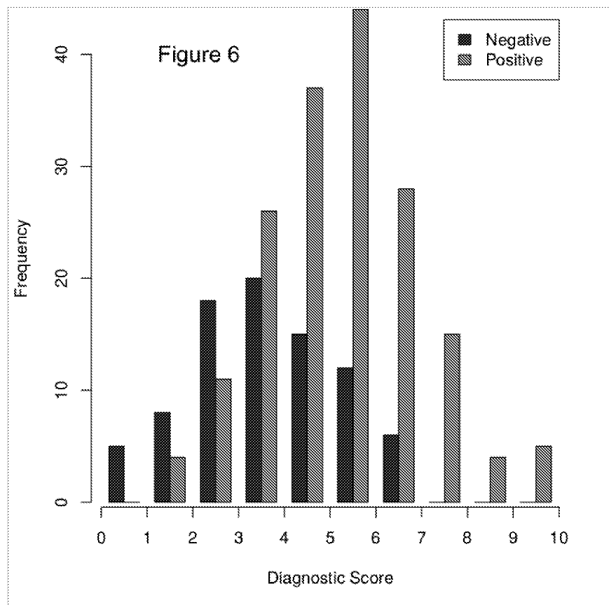

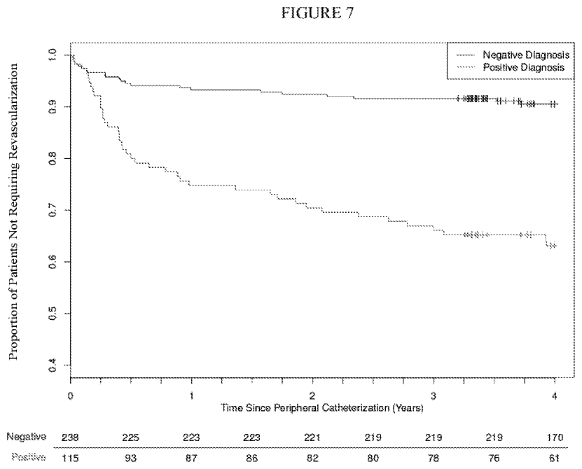

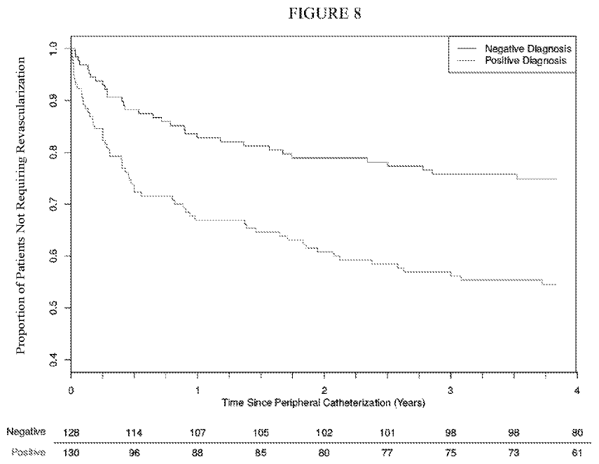

Once the final combination of protein markers and/or clinical variables was selected, a final model was built with the data from the entire population. Multivariable logistic regression evaluated the performance of the model in the population as a whole as well as in several relevant subgroups, to determine how well the model performed in men vs. women, and correcting for age. Diagnostic odds ratios (OR) with 95% confidence intervals (CI) were generated. We generated a score distribution within the population, followed by receiver operator characteristic (ROC) testing with valor of the score as a function of the AUC. Operating characteristics of the score were calculated, with sensitivity (Sn), specificity (Sp), positive and negative predictive value (PPV, NPV) generated. We also looked at methods for transforming the single diagnostic score into levels of likelihood (e.g., a five-level score, where a score of 1 means that the patient is extremely unlikely to have PAD or a negative result, a score of 3 means that the patient has an intermediate likelihood to have PAD or a positive result), and a score of 5 means that the patient is extremely likely to have PAD or a positive result), and evaluated each of these levels with the above operating characteristics (FIG. 5, Example #1) We also looked at methods for transforming the single diagnostic score into levels of likelihood (e.g., a ten-level score, where a score of 1 means that the patient is extremely unlikely to have PAD or a negative result, a score of 5 means that the patient has an intermediate likelihood to have PAD or a positive result, and a score of 10 means that the patient is extremely likely to have PAD or a positive result), and evaluated each of these levels with the above operating characteristics (FIG. 6, Example #2). Lastly, time to first peripheral revascularization (balloon, stent or bypass) event as a function of PAD elevated score was calculated, displayed as Kaplan-Meier survival curves, and compared using log-rank testing (FIG. 7, Example #1; FIG. 8, Example #2).

All statistics were performed using R software, version 3.3 or later (R Foundation for Statistical Computing, Vienna, AT); p-values are two-sided, with a value <0.05 considered significant.

Following the described methods, independent predictors of PAD ≥50% obstruction in any one vessel included six protein markers (angiopoietin 1, eotaxin 1, follicle stimulating hormone, interleukin 23, kidney injury molecule 1, and midkine) and one clinical variable (history of hypertension). This combination of protein markers and clinical variables is represented by panel PAD158, Example #1, as shown in Table 3 and FIGS. 1, 5 and 7.

In multivariable logistic regression, our score was strongly predictive of severe PAD in all subjects (OR=13.79, 95% CI 8.06-23.58; p<0.001).

We calculated individual scores and expressed results as a function of PAD presence. In doing so, a bimodal score distribution was revealed, with higher prevalence of severe PAD in those with higher scores, and lower prevalence among those with lower scores. We also calculated a 5-point score for diagnosis of peripheral artery disease and/or monitoring PAD progressions or therapeutic effect which demonstrated increasing stenosis with each higher score (FIG. 5). In ROC testing, for the gold standard diagnosis of >50% obstruction of any major peripheral artery, the scores generated had an in sample AUC of 0.85 (FIG. 1; p<0.001).

The clinical and protein marker scoring strategy disclosed herein can reliably diagnose the presence of peripheral artery disease (PAD). Advantages of a reliable clinical and protein marker score for diagnosing PAD presence include the fact such a technology can be widely disseminated in a cost-effective manner, easily interpreted, and are associated with a well-defined sequence of therapeutic steps.

Example 2: A Clinical and Protein Marker Scoring System to Diagnose Peripheral Artery Disease (PAD), Panel PAD076

In a prospective cohort of 258 patients referred for diagnostic peripheral angiography enrolled in the Catheter Sampled Blood Archive in Cardiovascular Diseases Study [71-72], predictors of ≥50% obstruction in at least one peripheral vessel were identified from over fifty clinical variables and 113 protein markers (selected due to plausible association to vascular disease) measured in blood obtained prior to angiography; protein markers were measured using the Luminex 100/200 xMAP technology platform (Myriad RBM, Austin, Tex.). Candidate predictive panels were created with least angle regression (LARS), and predictive models were generated using LASSO with logistic regression. Once the final combination of protein markers and/or clinical variables was selected, an algorithm derived from the final model was built with the entire population, and evaluated within the same population to predict PAD.

The final panel consisted of two clinical variables (body mass index, history of hypertension) and six protein markers (fetuin A, interleukin 8, kidney injury molecule 1, osteopontin, T Cell specific protein RANTES, and Tamm-Horsfall urinary glycoprotein). Notably, similar trends of each protein marker have individually been linked to cardiovascular disease development, vascular calcification and/or risk. [73-75]. The final had an in sample area under the receiver operating characteristic curve (AUC) of 0.76 for obstructive PAD (FIG. 2, Example #2). At optimal cut-off, the score had 63% sensitivity, 75% specificity, 84% positive predictive value (PPV) and 50% negative predictive value (NPV) for obstructive PAD. When the score was divided into low risk (score of ≤3/10) and high risk (score of ≥7/10) groups, we found NPV of 67% and PPV of 100% for obstructive PAD for each subgroup respectively (FIG. 6, Example #2). An elevated score predicted revascularization within 1 year follow up (age- and sex-adjusted hazard ratio: 2.1; p=0.00249); such risk extended to at least to 5 years (FIG. 8, Example #2).

Using an approach leveraging clinical information plus proteomic screening, we describe a novel method to predict angiographically significant PAD, also lending potential prognostic information regarding need for revascularization. The protein markers in this model all have plausible biologic links to atherosclerosis and/or vascular calcification. Clinically, use of a tool such as this could act as a gatekeeper prior to imaging or invasive testing. It may also be used to evaluate at-risk patients for risk of vascular complications; as such, a role in clinical trials to enrich for PAD-related events or to identify patients at risk for adverse effects of drug therapies is plausible.

Example 3: A Clinical and Protein Marker Scoring System for Prognosis of Peripheral Limb Amputation, Panel AMPU018

This example demonstrates yet another non-invasive method employing a clinical and protein marker scoring system that offers, among other things, high accuracy in providing a prognosis of limb amputation. This example utilized the same described methods (study design and participants, data acquisition, follow up, protein marker testing, statistics and results (Tables 1, 2, and 3 and FIG. 3) as Example 1. The primary differences between Example 1 and Example 3 are the subjects, clinical variables and proteins that were utilized and the outcome was prognosis of peripheral limb amputation.

Example 4: A Clinical and Protein Marker Scoring System to Diagnose Aortic Stenosis (AS), Panel ASR025

This example demonstrates yet another non-invasive method employing a clinical and protein marker scoring system that offers, among other things, high accuracy in diagnosing severe aortic stenosis.

This example utilized the same described methods as Examples 1, 2, and 3 (study design, data acquisition, follow up, protein marker testing, statistics and results), with the exception of the subjects, clinical variables, and proteins utilized and the outcome was diagnosis and/or monitoring of aortic stenosis (Tables 1, 2, and 3 and FIG. 4, Example #4).

Example 5: Further Demonstration of Methods Employing Clinical and Protein Marker Analysis for the Diagnosis Cardiovascular Diseases

Table 3 is a chart of the different panels comprising protein markers and optionally clinical variables with corresponding AUCs for the given outcome. These reflect aforementioned Examples 1 through 4, as well as additional panels generated using the methods and analysis provided herein.

TABLE 3
Performance of Different Panels for Various Outcomes
Comprising Protein Markers and Optionally Clinical Variables
with Corresponding AUCs and FIGS.

| | | | Cross | In | |
|---|---|---|---|---|---|
| | | | Validated | Sample/ | |
| | | | Mean | Entire | |
| | Test | Protein | AUCs | Population | |
| Analysis | Outcome/ | markers & | (rounded | (rounded | |
| # for | Positive | Clinical | to nearest | to nearest | FIG. |
| Panels | Endpoint | Variables | 0.00) | 0.00) | Reference |
| Diagnostic and/or | | | | | |
| Prognostic and/or | | | | | |
| Monitoring Progression | | | | | |
| and/or Therapeutic Effect | | | | | |
| PAD158 | Diagnosis | Angiopoietin 1, | 0.84 | 0.85 | 1, 5, 7 |
| Example 1 | for >50% | Eotaxin 1, Follicle | | | |
| | obstruction | Stimulating | | | |
| | in | Hormone, | | | |
| | peripheral | Interleukin 23, | | | |
| | arteries | Kidney Injury | | | |
| | and/or | Molecule 1, | | | |
| | monitoring | Midkine, History | | | |
| | PAD | of Hypertension | | | |
| | progression | | | | |
| | or | | | | |
| | therapeutic | | | | |
| | effect | | | | |
| PAD027VA | Diagnosis | Kidney Injury | 0.87 | 0.83 | |
| | for >50% | Molecule 1, | | | |
| | obstruction | Interleukin 1 | | | |
| | in | receptor | | | |
| | peripheral | antagonist, | | | |
| | arteries | Pulmonary | | | |
| | and/or | Surfactant | | | |
| | monitoring | Associated Protein | | | |
| | PAD | D, History of | | | |
| | progression | percutaneous | | | |
| | or | peripheral | | | |
| | therapeutic | angioplasty | | | |
| | effect | (with or without | | | |
| | | stent) | | | |
| PAD104 | Diagnosis | Serotransferrin, T | 0.84 | 0.87 | |
| | for >50% | Cell Specific | | | |
| | obstruction | Protein RANTES, | | | |
| | in | Thyroxine Binding | | | |
| | peripheral | Globulin, | | | |
| | arteries | Transthyretin | | | |
| | and/or | | | | |
| | monitoring | | | | |
| | PAD | | | | |
| | progression | | | | |
| | or | | | | |
| | therapeutic | | | | |
| | effect | | | | |
| PAD103 | Diagnosis | Serotransferrin, T | 0.84 | 0.90 | |
| | for >50% | Cell Specific | | | |
| | obstruction | Protein RANTES, | | | |
| | in | Thyroxine | | | |
| | peripheral | Binding Globulin, | | | |
| | arteries | Transthyretin, | | | |
| | and/or | History of | | | |
| | monitoring | Hypertension | | | |
| | PAD | | | | |
| | progression | | | | |
| | or | | | | |
| | therapeutic | | | | |
| | effect | | | | |
| PAD076 | Diagnosis | Fetuin A, | 0.72 | 0.76 | 2, 6, 8 |
| Example 2 | for >50% | Interleukin 8, | | | |
| | obstruction | Kidney Injury | | | |
| | in | Molecule 1, | | | |
| | peripheral | Osteopontin, T | | | |
| | arteries | Cell Specific | | | |
| | and/or | Protein RANTES, | | | |
| | monitoring | Tamm Horsfall | | | |
| | PAD | Urinary | | | |
| | progression | Glycoprotein, | | | |
| | or | Body Mass Index, | | | |
| | therapeutic | History of | | | |
| | effect | Hypertension | | | |
| PAD157 | Diagnosis | Angiopoietin 1, | 0.84 | 0.85 | |
| | for >50% | Eotaxin 1, Follicle | | | |
| | obstruction | Stimulating | | | |
| | in | Hormone, | | | |
| | peripheral | Interleukin 23, | | | |
| | arteries | Kidney Injury | | | |
| | and/or | Molecule 1, | | | |
| | monitoring | Midkine, History | | | |
| | PAD | of Dyslipidemia, | | | |
| | progression | History of | | | |
| | or | Hypertension | | | |
| | therapeutic | | | | |
| | effect | | | | |
| PAD154 | Diagnosis | Angiopoietin 1, | 0.84 | 0.86 | |
| | for >50% | Apolipoprotein Cl, | | | |
| | obstruction | Eotaxin 1, Follicle | | | |
| | in | Stimulating | | | |
| | peripheral | Hormone, | | | |
| | arteries | Interleukin 23, | | | |
| | and/or | Kidney Injury | | | |
| | monitoring | Molecule 1, | | | |
| | PAD | Matrix | | | |
| | progression | Metalloproteinase | | | |
| | or | 7, Midkine, Tamm | | | |
| | therapeutic | Horsfall Urinary | | | |
| | effect | Glycoprotein, | | | |
| | | History of | | | |
| | | Dyslipidemia, | | | |
| | | History of | | | |
| | | Hypertension | | | |
| PAD145 | Diagnosis | Angiopoietin 1, | 0.70 | 0.73 | |
| | for >50% | Eotaxin 1, Fetuin | | | |
| | obstruction | A, Interleukin 23, | | | |
| | in | Kidney Injury | | | |
| | peripheral | Molecule 1, | | | |
| | arteries | History of | | | |
| | and/or | Hypertension | | | |
| | monitoring | | | | |
| | PAD | | | | |
| | progression | | | | |
| | or | | | | |
| | therapeutic | | | | |
| | effect | | | | |
| PAD001 | Diagnosis | Angiotensin | 0.89 | 0.93 | |
| | for 50% + | Converting | | | |
| | obstruction | Enzyme, | | | |
| | in any | Carcinoembryonic | | | |
| | peripheral | antigen related cell | | | |
| | artery | adhesion molecule | | | |
| | and/or | 1, Interferon | | | |
| | peripheral | gamma Induced | | | |
| | intervention | Protein 10, | | | |
| | (balloon, | Osteopontin, | | | |
| | stent, or | Pulmonary | | | |
| | bypass) | surfactant | | | |
| | and/or | associated protein | | | |
| | monitoring | D, T Cell Specific | | | |
| | PAD | Protein RANTES, | | | |
| | progression | Tamm Horsfall | | | |
| | or | Urinary | | | |
| | therapeutic | Glycoprotein, | | | |
| | effect | History of | | | |
| | and/or | Peripheral | | | |
| | prognosis | revascularization | | | |
| | for | intervention | | | |
| | peripheral | (peripheral | | | |
| | limb | angioplasty, stent | | | |
| | amputation | or bypass), | | | |
| | | History of | | | |
| | | Hypertension | | | |
| PAD010 | Diagnosis | Angiotensin | 0.89 | 0.90 | |
| | fo r50% + | Converting | | | |
| | obstruction | Enzyme, Kidney | | | |
| | in any | Injury Molecule 1, | | | |
| | peripheral | Osteopontin, | | | |
| | artery | Pulmonary | | | |
| | and/or | surfactant | | | |
| | peripheral | associated | | | |
| | intervention | protein D, | | | |
| | (balloon, | History of | | | |
| | stent, or | Peripheral | | | |
| | bypass) | revascularization | | | |
| | and/or | intervention | | | |
| | monitoring | (peripheral | | | |
| | PAD | angioplasty, stent | | | |
| | progression | or bypass) | | | |
| | or | | | | |
| | therapeutic | | | | |
| | effect | | | | |
| | and/or | | | | |
| | prognosis | | | | |
| | for | | | | |
| | peripheral | | | | |
| | limb | | | | |
| | amputation | | | | |
| PAD026 | Prognosis | Angiotensin | 0.70 | 0.77 | |
| | for | Converting | | | |
| | peripheral | Enzyme, | | | |
| | intervention | ENRAGE, | | | |
| | (balloon, | Intercellular | | | |
| | stent, or | Adhesion Molecule | | | |
| | bypass) or | 1, Monokine | | | |
| | prognosis | Induced by | | | |
| | for | Gamma Interferon, | | | |
| | peripheral | Myeloid Progenitor | | | |
| | limb | Inhibitory | | | |
| | amputation | Factor 1, | | | |
| | | Pulmonary | | | |
| | | surfactant | | | |
| | | associated protein | | | |
| | | D, Resistin, Age | | | |
| PAD018 | Prognosis | Matrix | 0.73 | 0.77 | |
| | for peripheral | Metalloproteinase | | | |
| | intervention | 7, Matrix | | | |
| | (balloon, stent, | Metalloproteinase | | | |
| | or bypass) or | 9 Total, Myeloid | | | |
| | prognosis for | Progenitor | | | |
| | peripheral limb | Inhibitory | | | |
| | amputation | Factor 1, | | | |
| | | Pulmonary | | | |
| | | surfactant | | | |
| | | associated | | | |
| | | protein D, Resistin, | | | |
| | | History of | | | |
| | | Peripheral | | | |
| | | revascularization | | | |
| | | intervention | | | |
| | | (peripheral | | | |
| | | angioplasty, stent | | | |
| | | or bypass) | | | |
| AMPU018 | Prognosis for | Kidney Injury | 0.84 | 0.87 | 3 |
| Example 3 | Peripheral Limb | Molecule 1, Vitamin | | | |
| | Amputation | D Binding Protein, | | | |
| | | History of Diabetes | | | |
| | | Mellitus Type 2 | | | |
| AMPU010 | Prognosis for | Factor VII, Vitamin D | 0.84 | 0.87 | |
| | Peripheral Limb | Binding Protein, | | | |
| | Amputation | History of Diabetes | | | |
| | | Mellitus Type 2, | | | |
| | | Smoker | | | |
| AMPU008 | Prognosis for | Factor VII, Growth | 0.84 | 0.87 | |
| | Peripheral Limb | Hormone, Vitamin D | | | |
| | Amputation | Binding Protein, | | | |
| | | History of Diabetes | | | |
| | | Mellitus Type 2, | | | |
| | | Smoker | | | |
| AMPU013 | Prognosis | Factor VII, Ferritin, | 0.82 | 0.86 | |
| | for Peripheral | Growth Hormone, | | | |
| | Limb | Immunoglobulin M, | | | |
| | Amputation | Vitamin D Binding | | | |
| | | Protein, History of | | | |
| | | Diabetes Mellitus | | | |
| | | Type 2 | | | |
| Diagnostic for AS | | | | | |
| ASR025 | Diagnosis and | Fetuin A, N terminal | 0.74 | 0.76 | 4 |
| Example 4 | Monitoring of | prohormone of brain | | | |
| | Aortic Value | natriuretic peptide, | | | |
| | Stenosis | von Willebrand | | | |
| | | Factor, Age | | | |
| ASR001 | Diagnosis and | Adiponectin, | 0.79 | 0.82 | |
| | Monitoring of | Apolipoprotein(a), | | | |
| | Aortic Value | Interleukin 18, N | | | |
| | Stenosis | terminal prohormone | | | |
| | | of brain natriuretic | | | |
| | | peptide, Resistin, von | | | |
| | | Willebrand Factor, | | | |
| | | Age, History of | | | |
| | | Coronary | | | |
| | | Revascularization | | | |
| | | Intervention (coronary | | | |
| | | angioplasty, stent or | | | |
| | | bypass) | | | |
| ASR002 | Diagnosis and | Adiponectin, | 0.78 | 0.80 | |
| | Monitoring of | Apolipoprotein(a), | | | |
| | Aortic Value | Interleukin 18, N | | | |
| | Stenosis | terminal prohormone | | | |
| | | of brain natriuretic | | | |
| | | peptide, Resistin, von | | | |
| | | Willebrand Factor, | | | |
| | | Age | | | |
| ASR003 | Diagnosis and | Apolipoprotein(a), N | 0.78 | 0.80 | |
| | Monitoring of | terminal prohormone | | | |
| | Aortic Value | of brain natriuretic | | | |
| | Stenosis | peptide, Resistin, von | | | |
| | | Willebrand Factor, | | | |
| | | Age | | | |
| ASR016 | Diagnosis and | Apolipoprotein(a), N | 0.77 | 0.79 | |
| | Monitoring of | terminal prohormone | | | |
| | Aortic Value | of brain natriuretic | | | |
| | Stenosis | peptide, von | | | |
| | | Willebrand Factor, | | | |
| | | Age | | | |
| ASR004 | Diagnosis and | N terminal | 0.77 | 0.78 | |
| | Monitoring of | prohormone of brain | | | |
| | Aortic Value | natriuretic peptide, | | | |
| | Stenosis | Resistin, von | | | |
| | | Willebrand Factor, | | | |
| | | Age | | | |
| ASR013 | Diagnosis and | Adiponectin, N | 0.76 | 0.77 | |
| | Monitoring of | terminal prohormone | | | |
| | Aortic Value | of brain natriuretic | | | |
| | Stenosis | peptide, von | | | |
| | | Willebrand Factor, | | | |
| | | Age | | | |
| ASR006 | Diagnosis and | N terminal | 0.75 | 0.77 | |
| | Monitoring of | prohormone of brain | | | |
| | Aortic Value | natriuretic peptide, | | | |
| | Stenosis | Osteopontin, von | | | |
| | | Willebrand Factor, | | | |
| | | Age | | | |

Example 6: Mathematical Determinations

A diagnostic or prognostic algorithm in the form of a linear model is represented by a mathematical formula in the following form:

Diagnostic score=a+b₁x₁+b₂x2+. . . +bₙxₙ where x₁ through xₙ are the model inputs (such as protein concentrations or clinical information), b₁ through bₙ are the coefficients of the model, and a is the “intercept” term.

Here is an example of a diagnostic algorithm in the form of a linear model, involving three protein concentrations as inputs:

Diagnostic score=3.5+1.8x₁+2.9x₂−1.3x₃

In this case, proteins 1 and 2 have a positive effect on disease risk (higher concentrations result in higher risk), as the coefficients are positive (as indicated by the plus sign in the model preceding the coefficients). Protein 3 has an inverse effect on disease risk (lower concentrations results in higher risk), as the coefficient is negative (as indicated by the minus sign preceding the coefficient).

If a patient has concentrations of 0.5 (protein 1), 2.5 (protein 2) and 1.5 (protein 3), then those concentrations are entered into the model and get the following:

Diagnostic score=3.5+(1.8*0.5)+(2.9*2.5)−(1.3*1.5)=9.7

The model would have cut-offs that would enable one to place 9.7 as either positive, intermediate, or negative result and allow for a determination of a diagnosis (or prognosis) of an outcome or event.

### REFERENCES

1. Norman, P E et al. Med. J. Aust. 2004, 181, 150-154;

2. Bergiers, S. et al. BMC Family Pract. 2011, 12, 39

3. Hirsch, A T et al. JAMA, 2001, 286, 1317-1324;

4. Allison, M A et al. Am. J. Prey. Med. 2007, 32, 328-333;

5. Selvin E. et al. Circulation 2004, 11, 738-743

6. Dohmen, A. Dtsch.Arztebl.Int.2012, 109, 95-101;

7. Norgren, L. Eur. J. Vasc. Surg.2007, 33(Suppl.S1), S1-S75

8. Fowkes F G et al., Lancet 2013, 382, 1329-1340;

9. Conte M S et al., J. Vasc. Surg. 2015, 61, S2-S41;

10. Hirsch A T et al., Vasc. Med. 2008, 13, 209-215

11. Adam D J et al., Lancet, 2005, 366, 1925-1934;

12. Lawall H. et al. Circulation 2006, 113, e463-e654; Norgren L. et al., J. Vasc. Surg. 2007, 45, S5-S67;

13. Varu V N et al. J. Vasc. Surg. 2010, 51, 230-241;

14. Lau J F et al, Nat. Rev. Cardiol. 2011,8, 405-418

15. Cheshire N J et al., J. Vasc. Surg. 1992, 15, 167-174;

16. Albers M. et al, J. Vasc. Surg. 1992, 16, 54-59; Schuler, JJ et al., J. Vasc. Surg. 1984, 1, 160-170;

17. Santilli J D et al., Am. Fam. Physician 1999, 59, 1899-1908

18. Arnold T E. et al., Ann. Vasc. Surg. 2000, 14, 450-456;

19. Brumberg R S et al. J., Vasc. Surg. 2007, 46, 1160-1166

20. Davies M G et al., Methodist DeBakey Cardiovasc. J. 2012, 8, 20-27

21. Sigvant B. et al., J. Vasc. Surg. 2007, 45, 1185-1191;

22. Peacock J M et al., Prey. Chronic Dis. 2011, 8, A141

23. Dimitrow P P, Polskie Arch. Med. Wew. 2014, 124, (12), 723-730

24. Sambrook, et al., Molecular Cloning: A Laboratory Manual (3rd Edition, 2001)

25. Sambrook, et al., Molecular Cloning: A Laboratory Manual (2nd Edition, 1989)

26. Maniatis et al., Molecular Cloning: A Laboratory Manual (1982); Ausubel et al.

27. Current Protocols in Molecular Biology (John Wiley and Sons, updated July 2008)

28. Short Protocols in Molecular Biology: A Compendium of Methods from Current Protocols in Molecular Biology, Greene Pub. Associates and Wiley-Interscience

29. Glover, DNA Cloning: A Practical Approach, vol. I & II (IRL Press, Oxford, 1985)

30. Anand, Techniques for the Analysis of Complex Genomes, (Academic Press, New York, 1992)

31. Transcription and Translation (B. Hames & S. Higgins, Eds., 1984)

32. Perbal, A Practical Guide to Molecular Cloning (1984)

33. Harlow and Lane, Antibodies, (Cold Spring Harbor Laboratory Press, Cold Spring Harbor, N.Y., 1998)

34. Knowles, J W et al., Arterioscler. Thromb. Vasc. Biol. 2007, 27, 2058-2078

35. Subherwal S. et al., Eur. J. Prey. Cardiol. 2015, 22,317-325

36. Walker, T G et al., Tech. Vasc. Interv. Radiol. 2009, 12, 117-129

37. Otto C M, Kuusisto J, Reichenbach D D, et al. Characterization of the early lesion of ‘degenerative’ valvular aortic stenosis. Histological and immunohistochemical studies. Circulation. 1994; 90: 844-853

38. Carabello B A. Introduction to aortic stenosis. Circ Res. 2013; 113:179-185

39. Kohler et al., Nature, 256: 495 (1975)

40. U.S. Pat. No. 4,816,567

41. Clackson et al., Nature, 352: 624-628 (1991)

42. Marks et al., J. Mol. Biol., 222: 581-597 (1991)

43. U.S. Pat. No. 4,816,567

44. Morrison et al., Proc. Natl. Acad. Sci. USA, 81: 6851-6855 (1984)

45. Harlow and Lane, eds. (Antibodies: A Laboratory Manual (1988) Cold Spring Harbor Laboratory Press, Cold Spring Harbor, N.Y.)

46. Ling-Na Zheng et al., 2011, J. of Analytical Atomic Spectrometry, 26, 1233-1236

47. Vaudel, M., et al., 2010, Proteomics, Vol. 10:4

48. Pan, S., 2009 J. Proteome Research, February; 8(2):787-97

49. Westermeier and Marouga, 2005, Bioscience Reports, Vol. 25, Nos. ½

50. Carr and Anderson, 2008, Clinical Chemistry. 54:1749-1752

51. Aebersold and Mann, 2003, Nature, Vol. 422

52. US2008/0202927

53. US2014/0256573

54. US2016/0153980

55. WO2016/001795

56. US2008/0185295

57. US2010/0047901

58. US2010/0231242

59. US2011/0154648

60. US2013/0306491

61. US2010/0329929

62. US2013/0261009

63. Sorger, 2008, Nature Biotechnol. 26:1345-1346

64. Li et al., 2002, Mol. Cell. Proteomics 1.2:157

65. Hou et al., 2006, J. Proteome Res. 5(10):2754-2759

66. Li et al., 2001, Proteomicsl(8):975-986

67. Ramsey et al., 2003, Anal. Chem. 75(15):3758-3764

68. Armenta et al., 2009, Electrophoresis 30(7): 1145-1156

69. Lynch et al., 2004, Proteomics 4(6):1695-1702

70. Kingsmore et al., 2003, Curr. Opin. Biotechnol. 14(1):74-81)

71. Catheter Sampled Blood Archive in Cardiovascular Diseases Study (Clinical Trials. Gov NCT00842868)

72. Gaggin H K, IJC Metabolic & Endocrine, 2014, 5, 11-8

73. Ibrahim N E, J Am Coll Cardiol. 2017, 69(9), 1147-56

74. Speer M Y, J Exp. Med. 2002, 196(8), 1047-55

75. Rothenbacher D Arterioscler. Thromb. Vasc. Biol. 2006, 26(1), 194-9

Each of the above references is incorporated herein by reference in its entirety.

1. A method of determining peripheral artery disease in a subject, comprising:
(i) providing a biological sample from a subject suspected of having peripheral artery disease,
(ii) applying the biological sample to an analytical device to:
(a) detect the concentration of at least two protein markers in the sample;
(b) normalize said concentration of protein markers against a synthetic quantification standard; and
(c) transform the normalized protein marker concentrations into a score;
wherein the at least two protein markers are selected from those set forth in Table 1;
(iii) optionally, determining the status of at least one clinical variable for the subject, wherein the clinical variable is selected from those set forth in Table 2
(iv) calculating a diagnostic score using an algorithm based on the normalized, transformed concentrations of protein markers determined in step (ii) and, optionally, the status of the clinical variable(s) determined in step (iii);
(v) classifying the diagnostic score as a positive, intermediate, or negative result; and
(vi) determining peripheral artery disease in a subject as indicated by the diagnostic score.

2. The method of claim 1 further comprising treating the subject based on the positive, intermediate, or negative result, wherein the treatment comprises a therapeutic intervention regimen.

3. The method of claim 1 wherein the sample comprises plasma.

4. The method of claim 1 wherein the at least two protein markers are selected from, angiopoietin 1, apolipoprotein C-I, angiotensin converting enzyme, carcinoembryonic antigen related cell adhesion molecule 1, eotaxin 1, ENRAGE, fetuin A, follicle stimulating hormone, intercellular adhesion molecule 1, interferon gamma induced protein 10, interleukin 1 receptor antagonist, interleukin 8, interleukin 23, kidney injury molecule 1, matrix metalloproteinase 7, matrix metalloproteinase 9 Total, midkine, monokine induced by gamma interferon, myeloid progenitor inhibitory factor 1, osteopontin, pulmonary surfactant associated protein D, resistin, serotransferrin, Tamm Horsfall urinary glycoprotein, T cell specific protein RANTES, thyroxine binding globulin, and transthyretin; and
wherein the optional step (iii) comprises determining the status of at least one clinical variable selected from age, history of hypertension, history of peripheral percutaneous angioplasty (with or without stent), body mass index (BMI), history of dyslipidemia, and/or history of peripheral revascularization (peripheral angioplasty, stent or bypass).

5. The method of claim 1, wherein the at least two protein markers are angiopoietin 1, eotaxin 1, follicle stimulating hormone, interleukin 23, kidney injury molecule 1, and midkine and wherein the optional step (iii) comprises determining the status of history of hypertension.

6. The method of claim 1, wherein the diagnosis of peripheral artery disease in the subject comprises a diagnosis of 50% or greater obstruction in a peripheral artery.

7. The method of claim 1, wherein a positive diagnostic score in the subject facilitates a determination by a medical practitioner of the need for one or more interventions selected from ultrasound, administration of pharmacological agents, peripheral angiography, peripheral revascularization (peripheral angioplasty, stent or bypass), and avoidance of any drug with a known amputation risk.

8. The method of claim 1, wherein a negative diagnostic score in the subject facilitates a determination by a medical practitioner of the need for one or more interventions selected from ongoing monitoring and management of peripheral and coronary risk factors, and lifestyle modifications.

9. The method of claim 1, wherein an intermediate diagnostic score in the subject facilitates a determination by a medical practitioner of the need for one or more interventions selected from further testing, arterial brachial index (ABI) testing, and more frequent monitoring for risk factors.

10. A method of administering a therapeutic intervention to a subject suspected of having peripheral artery disease comprising:
(i) determining the subject's protein marker profile for a panel of protein markers comprising at least two protein markers selected from those set forth in Table 1;
(ii) optionally, determining the status of at least one clinical variable for the subject, wherein the clinical variable is selected from those set forth in Table 2;
(iii) assigning a score to the subject based on the protein marker profile in (i) and optionally the clinical value status in (ii) wherein the score is classified as positive, intermediate, and negative, said score algorithmically-derived from the normalized and mathematically transformed concentrations of protein markers in the subject's sample and optionally, the status of at least one clinical variable; and
(iv) administering to the subject a therapeutic intervention based on the positive, intermediate or negative score.

11. A method of detecting two or more protein markers in a subject having hypertension and/or that is suspected of having peripheral artery disease, the method comprising:
(i) selecting a subject that has hypertension and/or that is suspected of having peripheral artery disease;
(ii) providing a biological sample from the subject;
(iii) applying the biological sample to an analytical device, and
(iv) detecting the concentration of at least two protein markers from Table 1.

12. The method of claim 11 further comprising:
(v) calculating a diagnostic score based on the concentration of protein markers determined in step (iv);
(vi) classifying the diagnostic score as a positive, intermediate, or negative result; and
(vii) determining peripheral artery disease in a subject as indicated by the diagnostic score.

13. The method of claim 11, wherein the at least two protein markers are selected from angiopoietin 1, apolipoprotein C-I, angiotensin converting enzyme, carcinoembryonic antigen related cell adhesion molecule 1, eotaxin 1, ENRAGE, fetuin A, follicle stimulating hormone, intercellular adhesion molecule 1, interferon gamma induced protein 10, interleukin 1 receptor antagonist, interleukin 8, interleukin 23, kidney injury molecule 1, matrix metalloproteinase 7, matrix metalloproteinase 9 Total, midkine, monokine induced by gamma interferon, myeloid progenitor inhibitory factor 1, osteopontin, pulmonary surfactant associated protein D, resistin, serotransferrin, Tamm Horsfall urinary glycoprotein, T cell specific protein RANTES, thyroxine binding globulin, and transthyretin.

14. (canceled)

15. (canceled)

16. (canceled)

17. (canceled)

18. (canceled)

19. A panel for the diagnosis and/or prognosis of peripheral artery disease, comprising target-binding agents that bind at least two protein markers selected from those listed in Table 1, a synthetic standard, and optionally, at least one clinical variable selected from those set forth in Table 2.

20. A panel for the diagnosis of 50% or greater obstruction in a peripheral artery comprising target-binding agents for angiopoietin 1, eotaxin 1, follicle stimulating hormone, interleukin 23, kidney injury molecule 1, and midkine and the clinical variable of history of hypertension.

21. A diagnostic kit comprising a panel according to claim 19.

22. Use of the panel of claim 19 in the evaluation of a subject's positive, intermediate, or negative response to a therapeutic and/or intervention for peripheral artery disease.

23. A method of determining risk of peripheral limb amputation in a subject within a time period, comprising:
(i) providing a biological sample from a subject suspected of having a risk of peripheral limb amputation,
(ii) applying the biological sample to an analytical device, to:
(a) detect the concentration of at least two protein markers in the sample;
(b) normalize said concentration of protein markers against a synthetic quantification standard and
(c) transform the normalized protein marker concentrations;
wherein the at least two protein markers are selected from those set forth in Table 1;
(iii) optionally, determining the status of at least one clinical variable for the subject, wherein the clinical variable is selected from those set forth in Table 2;
(iv) calculating a prognostic score using an algorithm based on the transformed, normalized protein marker concentrations determined in step (ii) and, optionally, the status of the clinical variable(s) determined in step (iii);
(v) classifying the prognostic score as a positive, intermediate, or negative result; and
(vi) determining risk of peripheral limb amputation as indicated by the prognostic score.

24. (canceled)

25. (canceled)

26. (canceled)

27. (canceled)

28. (canceled)

29. (canceled)

30. (canceled)

31. (canceled)

32. A method of administering a therapeutic intervention to a subject suspected of having a risk of peripheral limb amputation comprising:
(i) determining the subject's protein marker profile for a panel of protein markers comprising at least two protein markers selected from those set forth in Table 1;
(ii) optionally, determining the status of at least one clinical variable for the subject, wherein the clinical variable is selected from those set forth in Table 2;
(iii) assigning a score to the subject based on the protein marker profile in (i) and optionally the clinical value status in (ii) wherein the score is classified as positive, intermediate, and negative, said score algorithmically- derived from the normalized, mathematically transformed concentrations of protein markers in the subject's sample and optionally, the status of at least one clinical variable; and
(iv) administering to the subject a therapeutic intervention based on the positive, intermediate or negative score.

33. A method of detecting two or more protein markers in a subject having diabetes mellitus type 2 and/or that is suspected of having a risk of peripheral limb amputation, the method comprising:
(i) selecting a subject that has diabetes mellitus type 2 and/or that is suspected of having a risk of peripheral limb amputation;
(ii) providing a biological sample from the subject;
(iii) applying the biological sample to an analytical device, and
(iv) detecting the concentration of at least two proteins markers from Table 1.

34. (canceled)

35. (canceled)

36. (canceled)

37. (canceled)

38. (canceled)

39. (canceled)

40. (canceled)

41. (canceled)

42. A method of determining aortic valve stenosis in a subject, comprising:
(i) providing a biological sample from a subject suspected of having aortic valve stenosis,
(ii) applying the biological sample to an analytical device, to:
(a) detect the concentration of at least two protein markers in the sample;
(b) normalize said concentration of protein markers against a synthetic quantification standard and
(c) transform the normalized protein marker concentrations;
wherein the at least two protein markers are selected from those set forth in Table 1;
(iii) optionally, determining the status of at least one clinical variable for the subject,
wherein the clinical variable is selected from those set forth in Table 2;
(iv) calculating a diagnostic score using an algorithm based on the normalized, transformed concentration of protein markers determined in step (ii) and, optionally, the status of the clinical variable(s) determined in step (iii);
(v) classifying the diagnostic score as a positive, intermediate, or negative result; and
(vi) determining aortic valve stenosis in a subject as indicated by a positive diagnostic score.

43. (canceled)

44. (canceled)

45. (canceled)

46. (canceled)

47. (canceled)

48. (canceled)

49. (canceled)

50. A method of administering a therapeutic intervention to a subject suspected of having aortic valve stenosis comprising:
(i) determining the subject's protein marker profile for a panel of protein markers comprising at least two protein markers selected from those set forth in Table 1;
(ii) optionally, determining the status of at least one clinical variable for the subject, wherein the clinical variable is selected from those set forth in Table 2;
(iii) assigning a score to the subject based on the protein marker profile in (i) and optionally the clinical value status in (ii) wherein the score is classified as positive, intermediate, and negative, said score algorithmically- derived from the normalized, mathematically transformed concentrations of protein markers in the subject's sample and optionally, the status of at least one clinical variable; and
(iv) administering to the subject a therapeutic intervention based on the positive, intermediate or negative score.

51. A panel for the diagnosis, prognosis, and/or monitoring of aortic valve stenosis, comprising target-binding agents for at least two protein markers selected from those listed in Table 1 and optionally, at least one clinical variable selected from those set forth in Table 2.

52. (canceled)

53. A diagnostic kit comprising a panel according to claim 51.

54. Use of the panel of claim 51 in the evaluation of a subject's positive, intermediate, or negative response to a therapeutic and/or intervention for aortic valve stenosis.

55. A diagnostic kit comprising a panel according to claim 20.
